SEQUENCE LISTING (25 sequences)

SEQ ID NO: 1 - DNA, 3216 bp, Mus musculus
  source 1..3216: mol_type = other DNA
ccatttcttgtaccaacctactcctaactacaaccgaagttctttgtgccaggctcctatgtagactgcttcaccactccaagcaccttcagcatgcatgcggaagaaatatatacctctcttcagtgggacattcctacctcagaggcctctcagaagtgccaatcccctagcaaatgttcaggagcatggtgtgttgtgacgatgatttcctgtgtggtctgtatgggcttgttagcaacgtccattttcttgggcatcaagttcttccaggtatcctctcttgtcttggagcagcaggaaagactcatccaacaggacacagcattggtgaaccttacacagtggcagaggaaatacacactggaatactgccaagccttactgcagagatctctccattcaggtagtgactgcagcccttgtccacacaactggattcagaatggaaaaagttgttactatgtctttgaacgctgggaaatgtggaacatcagtaagaagagctgtttaaaagagggcgctagtctctttcaaatagacagcaaagaagaaatggagttcatcagcagtatagggaaactcaaaggaggaaataaatattgggtgggagtgtttcaagatggaatcagtggatcttggttctgggaagatggctcttctcctctctctgacttgttgccagcagaaagacagcgatcagccggccagatctgtggatacctcaaagattctactctcatctcagataagtgcgatagctggaaatattttatctgtgagaagaaggcatttggatcctgcatctgaaagatacaactcaaagctacctgttacacaagtgtttgaaagaggccaaagagcaaacacaggaggagattgatgtgagactatgaaggcaagcttgattttttatttacacttggcaatagtctgtaacgctttgcagatttcagttgaccttgtccattgtgccaaggaaaaaattgatttagagctacagaagatctgactcagtgagaacaaagaagagaaaataatttcctgtgtgtttgaaagtccaaagtgacgatccataccacaccgagatctagctgtccttgtgggagtttttatgcaatccttggtttgtacttttagtaaacaattctatctactatacttacaactgacaaaattttcttcaggtcaccttccagtaattatagatcataagatcacacactgagaggagagagatggatttctaaactgtagtattaccaccaatcatacgccaggggtcctttggctctaggtaaaaatgagcagaatggggacctccagtgtttttaatcttcttctgaggcccagggcctgggggatgacctcacagatatagagatttacagtttctaacccttggagctagggggctattgctccgggatctgagtgctcaggttgaaggcttatagcttttcgttttaatcactactggctctgggtcttggcagcttttacaatccctaacctttagcagcaataccagacagcagtcagtagcagcaagcagctcccactagtgttatagctcttggatggctttagggtggtggggcattaactctgcagtgactactattacagtcctctggtctagaatgcaggaatctcccatcagcccttgttggctgttagtgttacagctctcagcataggttttctgtgatccttggttccaaagactgcagctgcttggcactcttccaggtctccaagcccgtgtttctccttcctggttggccacagtcttctgtctttactcagccttgcttttctgccttctctcatttctctaactctaccattcctcagtctctctgtttctgctgcccatcctttctgccatgacccatctctggaatttttcaggtgttctcttgtctgtcgactgccacggtaactgctgtcaccactagagctgcttgctctgcttggttgcctttgctcccatcttgtgcttctgcatcttctctttgtgttagcagctcaggctactcttctaatgcagggcaaaggaggagaaaaaggaccattcgggaagtcttttattggtgttaatgttacagcatcaggagtgtcagaggagggggtgtcctggctgactctaaaactgaactaaacagcccatttaatagataagaagtgggtgtagtgcgggtgtctgcaccttcacagtagctcttcaaggactgtgtttgcaccaatcacagcacagactcttgtttacagtcacagatcttccctaggttcatggttggaagtgctccctctctggcccaaatgagaactgcaggacaggctgggccttgggcaatttgccaaaaggcttcccttgcttagttagagtgctgttttccataggctattccagttgtgggagcagtcacaactggagcatgtcttcagccatggggtggtggtattcttcagtagctagaggggcttcagagtaaagtgcccaaactgctggctggactgtcatgagggcagtctggccagatgttagtaaaaaggctgcttggctgatagcttgaattcacgttcacagacaataaaagtgtgtttacaataaatacttaataaaaccccagtacatgacaattcgaatagcacttagcagtacttcataaaaatgaacatttaacagggtttaacttctgccccaaagctgtagagtcattgagtccctctgatctgcctcagaaggaaccagggctttttctgtctatggtgcttttcagactgcagagttctgctgggaaaggtactctttgaatttcatgtaggggaaggattagcatggacatcagcatgtcccattgtgtaggtggagccttccatcctattgctcacagatcaaacggctcagtctgcgctgacaagatgaacaagatgaacaacctagttcctctaactgcctgtgtctcaaccttcctgctataaaactgtaatacaattcacttcaaaatactgcttaagaacaccatccaaatattacatgcaaactataaaaaatgatactttagataagaaagggtaaatgaaaatgagtgctatctaccagaagaaaaattagctaattatagttatcatgttccgtacttataatgtgtcatctctaaatcatactacatacataacatgctcttaataaatatttgataaattcag

SEQ ID NO: 2 - protein, 238 aa, Mus musculus
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 3 - DNA, 1733 bp, Homo sapiens
  source 1..1733: mol_type = other DNA
gtacccggctgtggcaatctgtggtgaagttgaaatgcttttctgctagactggcaacatgttttgattcttctcaaataactcgcaagctcttgtgaagcacaacggcctaccttgtcctgaagaaaacatactctaaggcccacagatgattaaacccagccatttatgtgacaatagcatttttatttttcttatcaagatctgggtatttgatccacacaatggtaaagattcccatgttagccaagatctaaatgattcaaactgatgatactctctgcagaagaggtgactataaacgcagcccctgtgatgccaactggacatattatggagatagctgctatgggttcttcaaacacaacttgacatggaaagagagtgagcagtactgcactgacatgaaggctactctcctgaagactgacaaccagaacattctgtggatagaagtcaagggctccaatgtctcccaagacgaaagaatcccaagtctcatttggaggaatgctatcactaactaggattcctggagctaaaacattataggagttacttgttccagcctcctgtgtggactgctttcctatcaaagcaccttagacatgcacgaggaagaaatatacacctctcttcagtgggatagcccagcaccagacacttaccagaaatgtctgtcttccaacaaatgttcaggagcatgctgtcttgtgatggtgatttcatgtgttttctgcatgggattattaacagcatccattttcttgggcgtcaagttgttgcaggtgtccaccattgcgatgcagcagcaagaaaaactcatccaacaagagagggcactgctaaactttacagaatggaagagaagctgtgcccttcagatgaaatattgccaagccttcatgcaaaactcattaagttcagcccataacagcagtccttgtccaaacaattggattcagaacagagaaagttgttactatgtctctgaaatttggagcatttggcacaccagtcaagagaattgtttaaaggaaggttccacgctgctacaaatagagagcaaagaagaaatggattttatcactggcagcttgaggaagattaaaggaagctatgattactgggtggggttgtctcaggatggacacagcggacgctggctttggcaagatggctcctctccttctcctggcctgttgccagcagagagatcccagtcagctaaccaagtctgtggatacgtgaaaagcaattcccttctttcgtctaactgcagcacgtggaagtattttatctgtgagaagtatgcgttgagatcctctgtctgaaagaaattgtgttcaaagtgttctattacactgttatttggagcatgccattggaaaacccacccccaccccccctcaaaaaaacagaacagtaaaccaaaatgtgggccatgaaattagcaacctgggactcaataatacacttgggaatattcttccacaccgtccagatttcatttgatgttgttcacattgcaagagtaaaacttatttagagctacagaagacaaaaccctgaagagttaagaacaaacgcaaggaaataatttttattgtttaaagcccggaatgactgtaactttcacacaaggtacgcatctatgtttttgggggaggtgatgtagttacagctgactaatatttttaaaataaataaataaatttggcctttaaaactca

SEQ ID NO: 4 - protein, 241 aa, Homo sapiens
MHEEEIYTSLQWDSPAPDTYQKCLSSNKCSGACCLVMVISCVFCMGLLTASIFLGVKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 5 - DNA, 9850 bp, synthetic construct
  misc_feature 1..9850: note = Polynucleotide; Humanization Clec9a genomic fragment
  source 1..9850: mol_type = other DNA
ggtaaattataccttcattctcatggatgcttccttttatctgtgctgggagattgctttttattatatatttaatgaagaatgctcgcccttcttatagtgttgcaggtgtccaccattgcgatgcagcagcaagaaaaactcatccaacaagagagggcactgctaaactttacagaatggaagagaagctgtgcccttcagatgaaatattgccaagccttcatgcaaaactcattaagttcaggtaatggataaaaatcagtttccactgtatacatctaaatatatacaccaatacctcaaaagaaacagtgacacacattttgttctctgcaaaactgttaggattttataataagagtgacataattttggtcactcttcagttcaatcttatttataacatccaatactacgtggaaacactagggatccaaaagaatgcaactaaaggaaatgtacagtcaaatgccaaagaggtgtgtgtgcaattagtaggtcagattaaaaaaatacatgcacaaaaaaaaatcgttttatccagccattcaaatttgtaaggatgttttagttctagctccaaatttcccaatatatttagtttagatattgttttagattcaagtttagatagctttgagcaaaaaaattcattttgaaaatgagagtatttaccacaaaattcttttacgatgcaggacctagctcttttttaaaattctatgaagttactgcagtttaatcttttatatgctttacaagcattttaaacctaatgcagagtctatttctacctgtcatatagtttgccatgtattctatgcttggcatttcataatgcttgtacatatagtttagagaattaactttcctagactggataccatttacataattgccaatgagagaagtcaaacatcaaccatagttccttctgattaaaatttaatcgagtgtttgaaggatcactgtgtgcccaaccatgtttctctatgcagcaggccattgcaaactcgggtgaggcccacctctgagccatcaaccttgaatagaataacaatatccattttggagacgtacaagtccaacatgttcttcaacgccaccaataccattgtctacactatttatactatagcaatgttcaggatattggttggcagggatttcaaacaaaagatattgaagagctttaaacaacttaacatctggcccatggaagctactgatgaattatttcttggcctaaatacagggactgtctagaaaccagagttgtggaagtttcagtgtctttccttctataaacctgtgccagaaacacttggtcaaattcttggtctttggttccccttttacacatttgaccatccttcttttgtgactatattccatcttaaaacaggttccataaaactgattaaacattaaggcttacttcttagttgtatcagagtcctaaactaagcatattgttcatttatgttcttctccaattttcctcacctgcatgttttcaaatcactccttaaggcagactacttgttctctccattatctataaaatatttccttgtttagctctcagaatttaaacataaattcagggcttattattcaatccatccaatggtccagtacatttctggctaaattggggcagtaagactaacagacatgaaacaataagcaaacaagtgaagacaataaatgatgtgaattcaaagaaaggaaagattgataaaactggaataatcagattattatagaagagaaaaaatttagttaaatatatcccaaaattaatttcatatttaagttaaaggcccattgcagatagaaattattacatgaatcttactatattgagcctcagagaagctgaaattggtaagtgctacatcatgtggaccattacgttatgagattcagcctcactgagggtgaggcaaggggctttttggagggatatatgttgttaatattttacaataagatactaaagtaaaatgtttatattcaaagcccataacagcagtccttgtccaaacaattggattcagaacagagaaagttgttactatgtctctgaaatttggagcatttggcacaccagtcaagagaattgtttaaaggaaggttccacgctgctacaaatagagagcaaagaagaaatggtaaacactgttttgtggtctcatgttattctgaaaatatcattttacttattaaaagaaatccctcattctcataagacaaaattccttaatattctataacagcagtataattgtaaaaataaaggtttaaaagaaacaaagttggaatctgtattttttgttttctttttaatgggaaaacataaaaacaggatggttttctgaaatcgagaagaatatgtttgttctatgtccttggttaattaaaaagaggagggaataaggagaatactattttgtagcatgataatccacttcaagatatacatatattatagttgcagatatgttttaaatgtattattttcaatgctgtaggcaaatatagtaacctataataatcttgtaattatcaagaaaaaacagaagtataaaaactgaagtggtgccatgaaataaataggtcttgaaaaatacaagtatctgctaaatgaaatgtttaattagcataaaacctagtttaccaatatcttaacaagtgaataggctgggcggggtggctcacacctgtaatcccagcattttgggaggccaaggtgggtggatcacctgagttcaaggccagcctggccaacacggtgaaaccctgtctctactaaaaatacaaaaattaggctgggatggtggtgggagcctgtaattccagctacgtgggaggctgaggcaggagaattacttgaacccaggaggtagaggtttgcaatgagccgagattcagccattgcactccagcttgggcaacaaaagcaaaactccatctcaaaaaaataataataataaaataaaacaagtgaacaataagatgttgccttgaattcattctacccacaaatatatgaaatagaatgcaaaattgcacatttttggccctataaaaaatgtatacctgctatattcagtaattagatgaagttcatttcaacaaaaatcatgagaaaataatttgttaaggatatgcagtaaaccaagaacatttgaagtaaattattacagttgtaggtgcaaaaagttattttacgtgcaaatttgatgacacaaattagaaatgcttcttctaaccgaacgtgctcatagttaagttgctcctggaatctcatgaactatttttctacatcgacgtatactgagaaagactattctatgtggtctatgatgtcaagaatatttttatttttgataacgaaggttcagtataacactttctttttttgacgtatactacctgccaggctctgaatctgttgaaacttttaaaatcccagttgcctatttttttcatagtttttaacaccacatggatccatacagcaatcgaaaatagtctaaaaaatgtatcggcacttttttataatactcccttctctccttttccagatttcaagttcccattaatttctagctaggcaaaatgtcacactttatatttcactagagttttgtttgtttgtttttctcatttcacagttcaatttttgtctcataggattttatcactggcagcttgaggaagattaaaggaagctatgattactgggtggggttgtctcaggatggacacagcggacgctggctttggcaagatggctcctctccttctcctggcctgtaagtctctgagtgaaatgctacaagaaaagttagaaacatgagggaattcaaaagtttatttcaccttgtaccatgaattttctccaagggacaagcagcatgataatgttacaggcggctaattgggattaaagctaacaaaattaagagtagcttcatgccttctaagtctctctccttcccagccctgcttctattatcttatagaaaattcacctcacaatttgagtgtctgatgaataatataattatcaagtgtgtttcttttgtatcgcttaatgtttagagaataactatttatttccacattaagcttttcagatggcagtggggagatggagaagcagaatcaggtgccttcatattttgaaaattaagagtcagcaaagagattctaatttagggactgtctttgggtatctatgttttctttttctttacatgctgtttttgttcaaattatattaacattctttattgagagaaagaagcagttactgaagaaggaggatgaaaccaccacaggaagttgctgtgtcaacacaaaacaaaacaagcagctacacacctctcattagttaccctcaggagcatttctgaaactcccaagtctaacctcagaaatgttgacttgcttttccaggttgccagcagagagatcccagtcagctaaccaagtctgtggatacgtgaaaagcaattcccttctttcgtctaactgcagcacgtggaagtattttatctgtgagaagtatgcgttgagatcctctgtctgaaagaaattgtgttcaaagtgttctattacactgttatttggagcatgccattggaaaacccacccccaccccccctcaaaaaaacagaacagtaaaccaaaatgtgggccatgaaattagcaacctgggactcaataatacacttgggaatattcttccacaccgtccagatttcatttgatgttgttcacattgcaagagtaaaacttatttagagctacagaagacaaaaccctgaagagttaagaacaaacgcaaggaaataatttttattgtttaaagcccggaatgactgtaactttcacacaaggtacgcatctatgtttttgggggactcgagataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggtaaatataaaatttttaagtgtataatgatgttaaactactgattctaattgtttgtgtattttaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggcggccggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctaggtaactataacggtcctaaggtagcgagctagcaagatacaactcaaagctacctgttacacaagtgtttgaaagaggccaaagagcaaacacaggaggagattgatgtgagactatgaaggcaagcttgatt

SEQ ID NO: 6 - DNA, 5118 bp, synthetic construct
  misc_feature 1..5118: note = Polynucleotide; Humanization Clec9a genomic fragment after cassette deletion
  source 1..5118: mol_type = other DNA
ggtaaattataccttcattctcatggatgcttccttttatctgtgctgggagattgctttttattatatatttaatgaagaatgctcgcccttcttatagtgttgcaggtgtccaccattgcgatgcagcagcaagaaaaactcatccaacaagagagggcactgctaaactttacagaatggaagagaagctgtgcccttcagatgaaatattgccaagccttcatgcaaaactcattaagttcaggtaatggataaaaatcagtttccactgtatacatctaaatatatacaccaatacctcaaaagaaacagtgacacacattttgttctctgcaaaactgttaggattttataataagagtgacataattttggtcactcttcagttcaatcttatttataacatccaatactacgtggaaacactagggatccaaaagaatgcaactaaaggaaatgtacagtcaaatgccaaagaggtgtgtgtgcaattagtaggtcagattaaaaaaatacatgcacaaaaaaaaatcgttttatccagccattcaaatttgtaaggatgttttagttctagctccaaatttcccaatatatttagtttagatattgttttagattcaagtttagatagctttgagcaaaaaaattcattttgaaaatgagagtatttaccacaaaattcttttacgatgcaggacctagctcttttttaaaattctatgaagttactgcagtttaatcttttatatgctttacaagcattttaaacctaatgcagagtctatttctacctgtcatatagtttgccatgtattctatgcttggcatttcataatgcttgtacatatagtttagagaattaactttcctagactggataccatttacataattgccaatgagagaagtcaaacatcaaccatagttccttctgattaaaatttaatcgagtgtttgaaggatcactgtgtgcccaaccatgtttctctatgcagcaggccattgcaaactcgggtgaggcccacctctgagccatcaaccttgaatagaataacaatatccattttggagacgtacaagtccaacatgttcttcaacgccaccaataccattgtctacactatttatactatagcaatgttcaggatattggttggcagggatttcaaacaaaagatattgaagagctttaaacaacttaacatctggcccatggaagctactgatgaattatttcttggcctaaatacagggactgtctagaaaccagagttgtggaagtttcagtgtctttccttctataaacctgtgccagaaacacttggtcaaattcttggtctttggttccccttttacacatttgaccatccttcttttgtgactatattccatcttaaaacaggttccataaaactgattaaacattaaggcttacttcttagttgtatcagagtcctaaactaagcatattgttcatttatgttcttctccaattttcctcacctgcatgttttcaaatcactccttaaggcagactacttgttctctccattatctataaaatatttccttgtttagctctcagaatttaaacataaattcagggcttattattcaatccatccaatggtccagtacatttctggctaaattggggcagtaagactaacagacatgaaacaataagcaaacaagtgaagacaataaatgatgtgaattcaaagaaaggaaagattgataaaactggaataatcagattattatagaagagaaaaaatttagttaaatatatcccaaaattaatttcatatttaagttaaaggcccattgcagatagaaattattacatgaatcttactatattgagcctcagagaagctgaaattggtaagtgctacatcatgtggaccattacgttatgagattcagcctcactgagggtgaggcaaggggctttttggagggatatatgttgttaatattttacaataagatactaaagtaaaatgtttatattcaaagcccataacagcagtccttgtccaaacaattggattcagaacagagaaagttgttactatgtctctgaaatttggagcatttggcacaccagtcaagagaattgtttaaaggaaggttccacgctgctacaaatagagagcaaagaagaaatggtaaacactgttttgtggtctcatgttattctgaaaatatcattttacttattaaaagaaatccctcattctcataagacaaaattccttaatattctataacagcagtataattgtaaaaataaaggtttaaaagaaacaaagttggaatctgtattttttgttttctttttaatgggaaaacataaaaacaggatggttttctgaaatcgagaagaatatgtttgttctatgtccttggttaattaaaaagaggagggaataaggagaatactattttgtagcatgataatccacttcaagatatacatatattatagttgcagatatgttttaaatgtattattttcaatgctgtaggcaaatatagtaacctataataatcttgtaattatcaagaaaaaacagaagtataaaaactgaagtggtgccatgaaataaataggtcttgaaaaatacaagtatctgctaaatgaaatgtttaattagcataaaacctagtttaccaatatcttaacaagtgaataggctgggcggggtggctcacacctgtaatcccagcattttgggaggccaaggtgggtggatcacctgagttcaaggccagcctggccaacacggtgaaaccctgtctctactaaaaatacaaaaattaggctgggatggtggtgggagcctgtaattccagctacgtgggaggctgaggcaggagaattacttgaacccaggaggtagaggtttgcaatgagccgagattcagccattgcactccagcttgggcaacaaaagcaaaactccatctcaaaaaaataataataataaaataaaacaagtgaacaataagatgttgccttgaattcattctacccacaaatatatgaaatagaatgcaaaattgcacatttttggccctataaaaaatgtatacctgctatattcagtaattagatgaagttcatttcaacaaaaatcatgagaaaataatttgttaaggatatgcagtaaaccaagaacatttgaagtaaattattacagttgtaggtgcaaaaagttattttacgtgcaaatttgatgacacaaattagaaatgcttcttctaaccgaacgtgctcatagttaagttgctcctggaatctcatgaactatttttctacatcgacgtatactgagaaagactattctatgtggtctatgatgtcaagaatatttttatttttgataacgaaggttcagtataacactttctttttttgacgtatactacctgccaggctctgaatctgttgaaacttttaaaatcccagttgcctatttttttcatagtttttaacaccacatggatccatacagcaatcgaaaatagtctaaaaaatgtatcggcacttttttataatactcccttctctccttttccagatttcaagttcccattaatttctagctaggcaaaatgtcacactttatatttcactagagttttgtttgtttgtttttctcatttcacagttcaatttttgtctcataggattttatcactggcagcttgaggaagattaaaggaagctatgattactgggtggggttgtctcaggatggacacagcggacgctggctttggcaagatggctcctctccttctcctggcctgtaagtctctgagtgaaatgctacaagaaaagttagaaacatgagggaattcaaaagtttatttcaccttgtaccatgaattttctccaagggacaagcagcatgataatgttacaggcggctaattgggattaaagctaacaaaattaagagtagcttcatgccttctaagtctctctccttcccagccctgcttctattatcttatagaaaattcacctcacaatttgagtgtctgatgaataatataattatcaagtgtgtttcttttgtatcgcttaatgtttagagaataactatttatttccacattaagcttttcagatggcagtggggagatggagaagcagaatcaggtgccttcatattttgaaaattaagagtcagcaaagagattctaatttagggactgtctttgggtatctatgttttctttttctttacatgctgtttttgttcaaattatattaacattctttattgagagaaagaagcagttactgaagaaggaggatgaaaccaccacaggaagttgctgtgtcaacacaaaacaaaacaagcagctacacacctctcattagttaccctcaggagcatttctgaaactcccaagtctaacctcagaaatgttgacttgcttttccaggttgccagcagagagatcccagtcagctaaccaagtctgtggatacgtgaaaagcaattcccttctttcgtctaactgcagcacgtggaagtattttatctgtgagaagtatgcgttgagatcctctgtctgaaagaaattgtgttcaaagtgttctattacactgttatttggagcatgccattggaaaacccacccccaccccccctcaaaaaaacagaacagtaaaccaaaatgtgggccatgaaattagcaacctgggactcaataatacacttgggaatattcttccacaccgtccagatttcatttgatgttgttcacattgcaagagtaaaacttatttagagctacagaagacaaaaccctgaagagttaagaacaaacgcaaggaaataatttttattgtttaaagcccggaatgactgtaactttcacacaaggtacgcatctatgtttttgggggactcgagataacttcgtataatgtatgctatacgaagttatgctaggtaactataacggtcctaaggtagcgagctagcaagatacaactcaaagctacctgttacacaagtgtttgaaagaggccaaagagcaaacacaggaggagattgatgtgagactatgaaggcaagcttgatt

SEQ ID NO: 7 - protein, 241 aa, synthetic construct
  REGION 1..241: note = Polypeptide: humanized Clec 9a protein
MHAEEIYTSLQWDIPTSEASQKCQSPSKCSGAWCVVTMISCVVCMGLLATSIFLGIKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 8 - DNA, 120 bp, Mus musculus
  source 1..120: mol_type = other DNA
ctgtgctgggagattgctttttattatatatttaatgaagaatgctcgcccttcttatagtgttgcaggtgtccaccattgcgatgcagcagcaagaaaaactcatccaacaagagaggg

SEQ ID NO: 9 - DNA, 120 bp, Homo sapiens
  source 1..120: mol_type = other DNA
gtttaaagcccggaatgactgtaactttcacacaaggtacgcatctatgtttttgggggactcgagataacttcgtataatgtatgctatacgaagttatatgcatgccagtagcagcac

SEQ ID NO: 10 - DNA, 157 bp, Mus musculus
  source 1..157: mol_type = other DNA
tcgccttcttgacgagttcttctgaataacttcgtataatgtatgctatacgaagttatgctaggtaactataacggtcctaaggtagcgagctagcaagatacaactcaaagctacctgttacacaagtgtttgaaagaggccaaagagcaaacac

SEQ ID NO: 11 - DNA, 200 bp, Homo sapiens
  source 1..200: mol_type = other DNA
gtttaaagcccggaatgactgtaactttcacacaaggtacgcatctatgtttttgggggactcgagataacttcgtataatgtatgctatacgaagttatgctaggtaactataacggtcctaaggtagcgagctagcaagatacaactcaaagctacctgttacacaagtgtttgaaagaggccaaagagcaaacacag

SEQ ID NO: 12 - DNA, 20 bp, synthetic construct
  misc_feature 1..20: note = forward primer
  source 1..20: mol_type = other DNA
ggagcagcaggaaagactca

SEQ ID NO: 13 - DNA, 24 bp, synthetic construct
  misc_feature 1..24: note = Probe (BHQ) primer
  source 1..24: mol_type = other DNA
tccaacaggacacagcattggtga

SEQ ID NO: 14 - DNA, 23 bp, synthetic construct
  misc_feature 1..23: note = reverse primer
  source 1..23: mol_type = other DNA
gcttggcagtattccagtgtgta

SEQ ID NO: 15 - DNA, 19 bp, synthetic construct
  misc_feature 1..19: note = forward primer
  source 1..19: mol_type = other DNA
ggttgccagcagaaagaca

SEQ ID NO: 16 - DNA, 22 bp, synthetic construct
  misc_feature 1..22: note = Probe (BHQ) primer
  source 1..22: mol_type = other DNA
atcagccggccagatctgtgga

SEQ ID NO: 17 - DNA, 22 bp, synthetic construct
  misc_feature 1..22: note = reverse primer
  source 1..22: mol_type = other DNA
tccagctatcgcacttatctga

SEQ ID NO: 18 - DNA, 22 bp, synthetic construct
  misc_feature 1..22: note = forward primer
  source 1..22: mol_type = other DNA
attgcgatgcagcagcaagaaa

SEQ ID NO: 19 - DNA, 23 bp, synthetic construct
  misc_feature 1..23: note = Probe (BHQ) primer
  source 1..23: mol_type = other DNA
catccaacaagagagggcactgc

SEQ ID NO: 20 - DNA, 21 bp, synthetic construct
  misc_feature 1..21: note = reverse primer
  source 1..21: mol_type = other DNA
ggcacagcttctcttccattc

SEQ ID NO: 21 - DNA, 24 bp, synthetic construct
  misc_feature 1..24: note = forward primer
  source 1..24: mol_type = other DNA
gcaacctgggactcaataatacac

SEQ ID NO: 22 - DNA, 25 bp, synthetic construct
  misc_feature 1..25: note = Probe (BHQ) primer
  source 1..25: mol_type = other DNA
tgggaatattcttccacaccgtcca

SEQ ID NO: 23 - DNA, 24 bp, synthetic construct
  misc_feature 1..24: note = reverse primer
  source 1..24: mol_type = other DNA
actcttgcaatgtgaacaacatca

SEQ ID NO: 24 - DNA, 3253 bp, Rattus norvegicus
  source 1..3253: mol_type = other DNA
tccattttttttgtaccagcctactcctgactactaaagaagttctttgtgttgggctcctatgttgactgctttacctctccaagcaccttcagcatgcatgaggaagaaatatacacctctcttcagtgggacattccaacctcagaggcctctcagaagtgtccatcccttagcaaatgtccaggaacatggtgtattgtgacggtgatttcctgtgtggtctgtgtgggcttactagcagcatccattttcttgggcatcaagttctcccaggtgtcctctcttgtaatggagcagcgggaaaggctcatccgacaggacacagcattgctgaacctcacagagtggcagaggaaccatacactgcagttaaaaagctgccaagcctcactacaaagatctctccgttcaggcagtaactgcaacccttgtccaccgaactggattcagaatggaaaaagttgttactatgcctttgaccgctgggaaacgtggaacaacagtaagaagagttgtttaaaagagggcgatagtctccttcaaatagacagcaaagaagaaatggagtttatcaacctcagtatatggaagctcaaaggaggatatgaatactgggtgggagtgtttcaagatggacccagtggatcttggttttgggaagatggctcttctcctctctctgacttgttgccaacagacagacagctatcagccagccagatctgtggatacctcaaagaccatactctcatctcggataactgcagtaactggaaatattttatctgtgagaagaaggcatttggatcctgcatctgaaagatacaactcaaagctacctgctacacaagtgttcgaaagaggtcagagagcaaatgcaggaggaggttggtgtgaggcctacaaaggcaaacttgctttttcatatgaacttggcaatattctataatgctttgaaaatttcagttgaccttgtccattgtacccaggagaaaattgatttagggctacagaagacctgactcattgagaacaaggaagagaaaatcatttcccgtctgtttgaaagtccaaagtgactatccataccgcacagacatctagctgtctttgtaggagtttttacagtaatccctggtttgctttttttttagtaaacaattctacctactatacttacaactgaccaaggatcacagaatttctttactgagagcttttcttcagatcagcttccagtcatggtgtatcataagattacacactgagaggagagagatggaattctaaatggtagtgttaccagcaatcacatactagggggacttaggtgctagataaaaatgagcagaatggggcctccagtattggtaatattcttctgagactcaaggcctaggaggctgaaactcacggatatagagatttacagtttctaacccttggagctaggggattattgctctaggatctgaatgctcaggttaaaggcttatagcttttcgttttaatcagtactgactccagatcttgacagctttcacaatccctgacctttagctgtagccttagacagcagtcagtagcagcaggcagctcccactggtcttataattcttggatggctgtagggtgatgggcattaactctgcagtgaccactattacagtcctctgatctagaatgtgggaatctctcgtcagcctctgttggctgttagtgttgcagctctcagtatagattttctgtgatccttggtcctaaagactgcagctccttgacactctttcaggtccccaagcccatgtttctcctctctggtttgccacacagccttctgtgtttattcagacttgagtttctgccttctttcttcttgtctctgactctaccattccttggcatctctctgattcttctgccccacgctttctgtcatggcccctctctggaattcttcagctgttcttttgtctctcgactgccactgccactgccacagtggctgctgtcaccaccagagccgctcgcttgcttgcctttgcaaccatcttgtgcttctgcgtcttctctttgtgttaacggctcaggccactcctctaatgatgcagagcaaaagagccaagagaaagggccattcaggaagtcttttattggtcctaatgttgcagcttcaggagtgtcagaggagggggtgttctggctgactctaaagctgaaccaaacaggttattttatagataagaaggaggtgtgataagggtgtgtgtcccttcacagcagcccttcaaggactgtgtttgtaccatcacagcacagactactgtttagacagtcacaggtctttccctagggccgtgatcggaaatgcgccccctctggcctgagagagaacagctgcaggatctctgggtctgtgttgtttgccaagacttctctttccataggctgttttggtggtgtgagcagttccgactggagcctgtcttcagtcatggagtggtgtattcttcagcttctttacctcagagtaaaatggccaaactgctggactgtcattgagggcagtctggccagttgtttgtaaacaggctgcttggttggtagcttaaatcattcattcccagacattaaaagtgtgcttacaataaatacttaataaaaccccactgtattacaagtctaatagcatttgacagtacttaataaaaatgaacatttagcgggatttaacttctgccccaaagctgtagaatcattgaatccctctcatctgtctcagaaggaaacagggttctactctctgtggattgtgcttttcagactgcagagttctgctgggaaagaaactattagaatttcacgtaggtaaaggaataggctagacatctgcacgtcccgttgtgtaggcgggacctcccatcttattgctcacgaatcaaacggctctgtctgcactgacaggatgaactacctagcacttctaactgacggtccccgtgcctcaacttccctgctataaaactgtaatatgattcacttcaaaatatcgcttaagaatgtcatccaaatgctacacgctaactatagaaaatggtattttaggcaggaaagggaaaattaaaatgtgtgacatctaccagaagaaaaatttgcaaatgatcgttatcatgttcccatgacatagatatttcgtgtgtcatctctgtcatactgcatagcatgctcttaataaatatttgctaaattc

SEQ ID NO: 25 - protein, 241 aa, Rattus norvegicus
MHEEEIYTSLQWDIPTSEASQKCPSLSKCPGTWCIVTVISCVVCVGLLAASIFLGIKFSQVSSLVMEQRERLIRQDTALLNLTEWQRNHTLQLKSCQASLQRSLRSGSNCNPCPPNWIQNGKSCYYAFDRWETWNNSKKSCLKEGDSLLQIDSKEEMEFINLSIWKLKGGYEYWVGVFQDGPSGSWFWEDGSSPLSDLLPTDRQLSASQICGYLKDHTLISDNCSNWKYFICEKKAFGSCI

(19) United States
(12) Patent Application Publication
(10) Pub. No.: US 2023/0413791 A1
(43) Pub. Date: Dec. 28, 2023

(54) NON-HUMAN ANIMALS HAVING A HUMANIZED CLEC9A GENE

(71) Applicant: Regeneron Pharmaceuticals, Inc., Tarrytown, NY (US)

(72) Inventors: Sokol Haxhinasto, Brookfield, CT (US); Roberto Donnianni, New York, NY (US)

(21) Appl. No.: 18/340,917

(22) Filed: Jun. 26, 2023

Related U.S. Application Data
(60) Provisional application No. 63/355,948, filed on Jun. 27, 2022.

(51) Int. Cl. A01K 67/027; C07K 14/705; G01N 33/50

(52) U.S. Cl. CPC A01K 67/0278; C07K 14/7056; A01K 2267/0387; A01K 2227/105; G01N 33/5088

(57) ABSTRACT

Disclosed herein is a genetically modified rodent animal comprising a humanized Clec9a gene in its genome. Also disclosed herein are an isolated rodent tissue or cell including a rodent embryonic stem cell, a rodent embryo, a method of making the genetically modified rodent animal, and methods of using the genetically modified rodent animal.

### CROSS REFERENCE TO RELATED APPLICATION

This application claims the benefit of priority from U.S. Provisional Application No. 63/355,948, filed Jun. 27, 2022, the entire contents of which is incorporated herein by reference.

### INCORPORATION BY REFERENCE OF SEQUENCE LISTING

The Sequence Listing in the XMIL format, named as 39341_10900US01_SequenceListing.xml of 53 KB, created on Jun. 13, 2023 and submitted to the United States Patent and Trademark Office via Patent Center, is incorporated herein in its entirety by reference.

### BACKGROUND

C-type lectin domain family 9 member A (Clec9a) is a C-type lectin-like receptor and is expressed at the cell surface of dendritic cells and a small subset of monocytes as a glycosylated dimer. Clec9a can mediate endocytosis, but not phagocytosis. Clec9a possesses a cytoplasmic immunoreceptor tyrosine-based activation-like motif (“ITAM”) that is reported to recruit Syk kinase and induce proinflammatory cytokine production.

### SUMMARY

In some embodiments, disclosed herein is a genetically modified rodent animal comprising a humanized Clec9a gene in its genome, wherein the humanized Clec9a gene comprises a rodent Clec9a nucleic acid sequence and a human CLEC9A nucleic acid sequence, wherein the humanized Clec9a gene encodes a humanized Clec9a polypeptide comprising an ectodomain substantially identical to the ectodomain of a human CLEC9A protein.

In some embodiments, the humanized Clec9a protein comprises a cytoplasmic-transmembrane sequence substantially identical to the cytoplasmic-transmembrane sequence of a rodent Clec9a protein (e.g., an endogenous rodent Clec9a protein). In some embodiments, the humanized Clec9a protein comprises a cytoplasmic-transmembrane sequence identical to the cytoplasmic-transmembrane sequence of a rodent Clec9a protein (e.g., an endogenous rodent Clec9a protein).

In some embodiments, the human CLEC9A nucleic acid sequence in a humanized Clec9a gene encodes at least a substantial portion of the ectodomain of the human CLEC9A protein. In some embodiments, the human CLEC9A nucleic acid sequence in a humanized Clec9a gene encodes amino acids 57-241 of a human CLEC9A (e.g., the human CLEC9A as set forth in SEQ ID NO: 4). In some embodiments, the human CLEC9A nucleic acid sequence comprises exon 3 through the Stop codon in exon 6 of a human CLEC9A gene. In some embodiments, the human CLEC9A nucleic acid sequence comprises exon 3 through exon 6 (i.e., through the 3′ end of exon 6) of a human CLEC9A gene.

In some embodiments, the rodent Clec9a nucleic acid sequence in a humanized Clec9a gene comprises a nucleotide sequence of a rodent Clec9a gene that encodes at least a substantial portion of the cytoplasmic-transmembrane sequence of a rodent Clec9a protein (e.g., an endogenous rodent Clec9a protein). In some embodiments, the rodent Clec9a nucleic acid sequence comprises exon 1 and exon 2 of a rodent Clec9a gene (e.g., an endogenous rodent Clec9a gene).

In some embodiments, the humanized Clec9a gene comprises (i) exon 1 and exon 2 of a rodent Clec9a gene (e.g., an endogenous rodent Clec9a gene), and (ii) exon 3 through the Stop codon in exon 6, optionally exon 3 through exon 6 of a human CLEC9A gene. In some embodiments, the humanized Clec9a gene comprises the 3′ UTR of a rodent Clec9a gene. In some embodiments, the humanized Clec9a gene comprises (i) exon 1 and exon 2 of a rodent Clec9a gene (e.g., an endogenous rodent Clec9a gene), (ii) exon 3 through exon 6 of a human CLEC9A gene, and (iii) the 3′ UTR of a rodent Clec9a gene (e.g., an endogenous rodent Clec9a gene).

In some embodiments, a humanized Clec9a gene is operably linked to a rodent Clec9a promoter, such as an endogenous rodent Clec9a promoter.

In some embodiments, a humanized Clec9a gene is located at a locus other than an endogenous rodent Clec9a locus. In some embodiments, a humanized Clec9a gene is located at an endogenous rodent Clec9a locus.

In some of the embodiments where a humanized Clec9a gene is located at an endogenous rodent Clec9a locus, the humanized Clec9a gene is formed as a result of replacement of a rodent Clec9a genomic DNA at an endogenous rodent Clec9a locus with a human CLEC9A nucleic acid. In some embodiments, a humanized Clec9a gene is formed as a result of replacement of a rodent genomic DNA comprising a nucleotide sequence encoding at least a substantial portion of the ectodomain of the endogenous rodent Clec9a protein, with the human CLEC9A nucleic acid which encodes at least a substantial portion of the ectodomain of the human CLEC9A protein.

In some embodiments, the rodent animal is a mouse, and wherein the mouse genomic DNA being replaced comprises exon 3 through the Stop codon in exon 6 of the endogenous mouse Clec9a gene, and the human genomic DNA comprises exon 3 through the Stop codon in exon 6 (optionally exon 3 through exon 6) of a human CLEC9A gene.

In some embodiments, a rodent animal is heterozygous for a humanized Clec9a gene.

In some embodiments, a rodent animal is homozygous for a humanized Clec9a gene.

In some embodiments, a humanized Clec9a polypeptide is expressed on dendritic cells in a rodent animal from a humanized Clec9a gene.

In some embodiments, the rodent is a mouse or a rat.

In some embodiments, disclosed herein is an isolated rodent tissue or cell, whose genome comprises a humanized Clec9a gene described herein. In some embodiments, the rodent cell is a rodent embryonic stem cell. In some embodiments, the rodent cell is an egg or a sperm. In some embodiments, an isolated rodent tissue or cell is a mouse tissue or mouse cell, or a rat tissue or rat cell.

In some embodiments, disclosed herein is a rodent embryo comprising a rodent embryonic stem cell which comprises a humanized Clec9a gene described herein.

In some embodiments, disclosed herein is a method of making a genetically modified rodent. In some embodiments, the method comprises modifying a rodent genome to comprise a humanized Clec9a gene, wherein the humanized Clec9a gene comprises a rodent Clec9a nucleic acid sequence and a human CLEC9A nucleic acid sequence, and encodes a humanized Clec9a polypeptide comprising an ectodomain substantially identical with the ectodomain of a human CLEC9A protein; and making a rodent comprising the modified rodent genome.

In some embodiments, modifying a rodent genome comprises the steps of introducing a nucleic acid molecule comprising a human CLEC9A nucleic acid sequence into the genome of a rodent embryonic stem (ES) cell, obtaining a rodent ES cell in which the human CLEC9A nucleic acid sequence has integrated into an endogenous Clec9a locus to replace a rodent Clec9a genomic DNA thereby forming a humanized Clec9a gene, and generating a rodent animal from the obtained rodent ES cell. In some embodiments, the human CLEC9A nucleic acid sequence encodes at least a substantial portion of the ectodomain of a human CLEC9A protein. In some embodiments, the nucleic acid molecule introduced into the ES cell further comprises a 5′ homology arm and a 3′ homology arm flanking the human CLEC9A nucleic acid sequence, and wherein the 5′ and 3′ homology arms are homologous to nucleic acid sequences at the endogenous rodent locus flanking the rodent Clec9a genomic DNA to be replaced. In some embodiments, the humanized Clec9a gene is operably linked to a rodent Clec9a promoter, e.g., an endogenous rodent Clec9a promoter at the endogenous rodent Clec9a locus.

In some embodiments of the method, the rodent is a mouse or a rat.

In some embodiments, disclosed herein is a targeting nucleic acid construct, comprising a human CLEC9A nucleic acid sequence to be integrated into a rodent Clec9a gene at an endogenous rodent Clec9a locus, flanked by a 5′ nucleotide sequence and a 3′ nucleotide sequence that are homologous to nucleotide sequences at the rodent Clec9a locus, wherein integration of the human CLEC9A nucleic acid sequence into the rodent Clec9a gene results in a replacement of a rodent Clec9a genomic DNA with the human CLEC9A nucleic acid sequence thereby forming a humanized Clec9a gene, and wherein the human CLEC9A nucleic acid sequence encodes at least a substantial portion of the ectodomain of a human CLEC9A protein. In some embodiments of a targeting nucleic acid, the rodent is a mouse or a rat.

In some embodiments, disclosed herein is an in vitro method for generating a genetically modified rodent cell, comprising introducing into a rodent cell a targeting vector comprising a human CLEC9A nucleic sequence that encodes at least a substantial portion of the ectodomain of a human CLEC9A protein, flanked by rodent homology arms that mediate integration of the human CLEC9A nucleotide sequence into an endogenous rodent Clec9a locus, which results in replacement of a rodent Clec9a genomic DNA with the human CLEC9A nucleic acid sequence to form a humanized Clec9a gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

In some embodiments, disclosed herein is a method of assessing pharmacokinetic properties of a candidate drug, the method comprising administering the candidate drug to a rodent animal described herein, and performing one or more assays to determine the pharmacokinetic properties of the candidate drug in the rodent animal. In some embodiments, the candidate drug is an antibody that binds to human CLEC9A. In some embodiments, the candidate drug is an antibody that is capable of binding to human CLEC9A.

In some embodiments, disclosed herein is a method for screening or evaluating candidate drugs targeting human CLEC9A, the method comprising administering the candidate drug to a rodent animal described herein, and performing one or more assays to determine whether the candidate drug has an effect on the rodent animal, e.g., induces activation of immune cells such as T cells. In some embodiments, the one or more assays comprise an assay that measures T cell proliferation in the rodent animal. In some embodiments, the one or more assays comprise an assay that measures proliferation of CD4+ T cells in the rodent animal. In some embodiments, the one or more assays comprise an assay that measures proliferation of CD8+ T cells in the rodent animal. In some embodiments, the candidate drug comprises an antibody that binds to human CLEC9A. In some embodiments, the candidate drug comprises an antibody that binds to human CLEC9A, fused with one or more peptides recognized by MHC molecules on dendritic cells of the rodent animal. In some embodiments, the one or more peptides comprise OVA peptide I (“OTI”, amino acids 257-264 of ovalbumin), and/or OVA peptide II (“OTI”, amino acids 323-339 of ovalbumin).

### BRIEF DESCRIPTION OF DRAWINGS

FIG. 1A depicts an exemplary embodiment of a strategy for humanization of a mouse Clec9a gene. The mouse Clec9a gene and the human CLEC9A gene are represented by horizontal lines, with their exons being represented by boxes placed above the lines. A contiguous mouse Clec9a genomic fragment of 7138 bp at an endogenous mouse Clec9a locus, which includes exon 3 through the Stop codon in exon 6, is replaced by a human CLEC9A genomic fragment of 4840 bp that encompasses exon 3 through exon 6. As a result, the entire ectodomain (including the “CLECT-NK-receptor-like” domain) of mouse Clec9a (amino acids 57-238) is replaced with the ectodomain of human CLEC9A (amino acids 57-241), while preserving the N-terminal 31 amino acids (including the “ITAM-like” motif) and transmembrane domain (amino acids 32-56) of mouse Clec9a. See also FIG. 1D. The locations of primers and probes used in human gain of allele and mouse loss of allele assays to confirm correct targeting are also indicated (designated as 7764hTU, 7764hTD, 7764mTU and 7764mTD). The sequences of the primers and probes are set forth in Table 2.

FIG. 1B depicts, as an exemplary embodiment of, not to scale, a targeting nucleic acid construct, which comprises a humanization fragment comprising human CLEC9A exons 3-6, followed by a Neomycin cassette with LoxP sites, flanked by a mouse 5′ homology arm of 45.6 kb from BAC clone Rp23-248K14 (including exon 1 and exon 2 of mouse Clec9a) and a mouse 3′ homology arm of 112.6 kb (including the 3′ UTR of exon 6 and downstream sequence of mouse Clec9a). The targeting nucleic acid construct comprising the humanized Clec9a gene can be introduced into a mouse embryonic stem cell for targeted insertion into the mouse genome. The junctions (“A”, “B” and “C”) are indicated with their sequences provided.

FIG. 1C depicts, as an exemplary embodiment of a strategy for humanization of a mouse Clec9a locus, not to scale, a humanized Clec9a locus resulting from removal of the Neomycin cassette of a humanized Clec9a locus generated using the targeting construct described in FIG. 1B. The junctions (“A” and “D”) are also indicated with their sequences provided.

FIG. 1D shows an alignment of exemplary protein sequences of mouse Clec9a (SEQ ID NO: 2), human CLEC9A (SEQ ID NO: 4), and humanized (“7765 mutant”) Clec9a (SEQ ID NO: 7). The transmembrane segment (“TM”) is underlined; the ectodomain is identified by a solid box; and the CLECT-NK-receptor-like domain is indicated by a dotted line.

FIGS. 2A-2D illustrate detection of Clec9a mRNA in wild-type (“WT”) mice (without humanization of the endogenous Clec9a gene), mice heterozygous for the humanized Clec9a gene (“Het”), and mice homozygous for the humanized Clec9a gene (“HumIn”). RT-PCR was performed on bulk RNA derived from splenocytes using probes against murine or human Clec9a nucleotide sequences. 2A-2B: Analysis done with probes targeting exons 1-2 or exons 3-4 on WT, Het, or HumIn mice. 2C-2D: Analysis done with probes targeting exons 2-3 or exons 5-6 on WT, Het, or HumIn mice. n=3 per mouse genotype; Gapdh was used as internal control.

FIGS. 3A-3B illustrate that mClec9a expression was restricted to circulating dendritic cells (also referred to as “cDC1”) in wild type (“WT”) mice and mice heterozygous for the humanized Clec9a gene (“Het”). 3A: Gating strategy performed on mouse splenocytes. Cells were gated based on size, singlets and live cells. Dendritic cells were gated on CD11c⁺ MHC-II⁺ and further sub-gated on Xcr1⁺ and CD11b⁺. 3B: Plots depicting expression of mouse Clec9a (“mClec9a”) on WT, Het, or mice homozygous for the humanized Clec9a gene (“HumIn”) mice, respectively. FMO; fluorescence minus one. FIGS. 4A-4B illustrate that humanized Clec9a expression was restricted to cDC1 in mice heterozygous for the humanized Clec9a gene (“Het”) and mice homozygous for the humanized Clec9a gene (“HumIn”). 4A: Gating strategy performed on mouse splenocytes. Cells were gated based on size, singlets and live cells. Dendritic cells were gated on CD11c⁺ MHC-II⁺ and further sub-gated on Xcr1⁺ and CD11b⁺. 4B: Plots depicting expression of humanized Clec9a (hClec9a) on wild type (“WT”), Het, or HumIn mice, respectively. FMO; fluorescence minus one.

FIG. 5 illustrates that humanized Clec9a expression was restricted to cDC1 in mice heterozygous for the humanized Clec9a gene (“Het”) and mice homozygous for the humanized Clec9a gene (“HumIn”). The left panel shows dot plot depicting expression of humanized Clec9a on Xcr1+ cells. The right panels show histograms depicting the expression of humanized Clec9a (“hClec9a) in wild type (“WT”), Het, or HumIn mice. FMO; fluorescence minus one.

FIGS. 6A-6B illustrate that humanized Clec9a expression was restricted to cDC1. 6A: Gating on splenic CD19⁺ B cells (upper panel) showing lack of expression of murine or human CLEC9A shown in the histograms corresponding to wild-type (“WT”) mice, mice heterozygous for the humanized Clec9a gene (“Het”), and mice homozygous for the humanized Clec9a gene (“HumIn”). FMO; fluorescence minus one. 6B: Gating on splenic CD4⁺ and CD8⁺ T-cells (upper panel) showing lack of expression of mouse or humanized Clec9a shown in the histograms corresponding to WT, Het, or HumIn mice. FMO; fluorescence minus one.

FIGS. 7A-7B illustrate that Clec9a targeting by antibody-antigen fusion elicits strong CD4+ T-cell responses in vivo. 7A: Gating on Ki67+ OT-II tetramer+ T-cells in mice injected with an anti-human CLEC9 antibody fused to an OVA peptide, an Isotype antibody fused to the OVA peptide, PBS, a full length OVA, or the OVA peptide, wherein the OVA peptide contains OT-I and OT-II antigens. 2×10⁶ CFSE-labeled OT-II T-cells were transferred to mice and antibodies were dosed at 15, 7.5, and 3.75 ug one day later in addition to PBS, the full length OVA, or the OVA peptide. Spleens were harvested 72 hours post antibody injection and analyzed by flow cytometry. 7B: Bar graph depicting the absolute count of Ki67⁺ OT-II tetramer⁺ T-cells shown in FIG. 7A. #, &, and @ indicate statistical significance from Isotype-OVA peptide fusion at 15, 7.5, and 3.75 ug per mouse, respectively. Error bars indicate standard deviation; One-way ANOVA.

FIGS. 8A-8B illustrate that Clec9a targeting by antibody-antigen fusion elicits strong CD8 T-cell responses in vivo. (A) Gating on Ki67+ OT-I tetramer+ T-cells in mice injected with an anti-human CLEC9 fused to an OVA peptide, an Isotype antibody fused to the OVA peptide, PBS, a full-length OVA, or the OVA peptide, wherein the OVA peptide contains OT-I and OT-II antigens. 2×10⁶ CFSE-labeled OT-I T-cells were transferred to mice and antibodies were dosed with 15, 7.5, and 3.75 ug one day later in addition to PBS, the full-length OVA, or the OVA peptide. Spleens were harvested 72 hours post antibody injection and analyzed by flow cytometry. (B) Bar graph depicting the absolute count of Ki67⁺ OT-I tetramer⁺ T-cells shown in FIG. 8A. #indicates statistical significance from Isotype-OVA Peptide at 15 ug per mouse. Error bars indicate standard deviation. One-way ANOVA.

### DETAILED DESCRIPTION

Disclosed herein are rodents (such as, but not limited to, mice and rats) genetically modified to comprise a humanized Clec9a gene. Also disclosed herein are compositions (e.g., targeting vectors) and methods for making such genetically modified rodents. The rodents disclosed herein can be used, e.g., but not limited to, as an in vivo system for evaluating candidate compounds directed to human CLEC9A, such as anti-human Clec9A antibodies, to target dendritic cells for broad immune related indications, including but not limited to autoimmunity, infectious disease, and cancer, among others. Accordingly, also disclosed herein are methods of using a genetically modified rodent for assessing candidate compounds directed to human CLEC9A.

### Humanization of Rodent Clec9a

Clec9a is a C-type lectin-like receptor and is expressed at the cell surface of dendritic cells and a small subset of monocytes as a glycosylated dimer. Clec9a can mediate endocytosis, but not phagocytosis. Clec9a possesses a cytoplasmic immunoreceptor tyrosine-based activation-like motif (“ITAM”) at its N-terminal portion, which is a conserved sequence of four amino acids that is repeated twice and is believed to recruit Syk kinase and induce proinflammatory cytokine production. See, e.g., Huysamen t al., J Biol Chem 283 (24), 16693-16701 (2008), which is incorporated herein by reference in its entirety.

Exemplary Clec9a sequences, including nucleic acid and protein sequences for human, mouse, rat, and humanized (mouse-human hybrid) Clec9a, are disclosed in the Sequence Listing and summarized in Table 1. An alignment of a human CLEC9A, a mouse Clec9a, and humanized (mouse-human hybrid) Clec9a protein sequences is provided in FIG. D.

For simplicity, the numbering of the exons herein refers to the coding exons of an Clec9a gene. For example, exon 1 of an Clec9a gene refers herein to the first coding exon of the Clec9a gene.

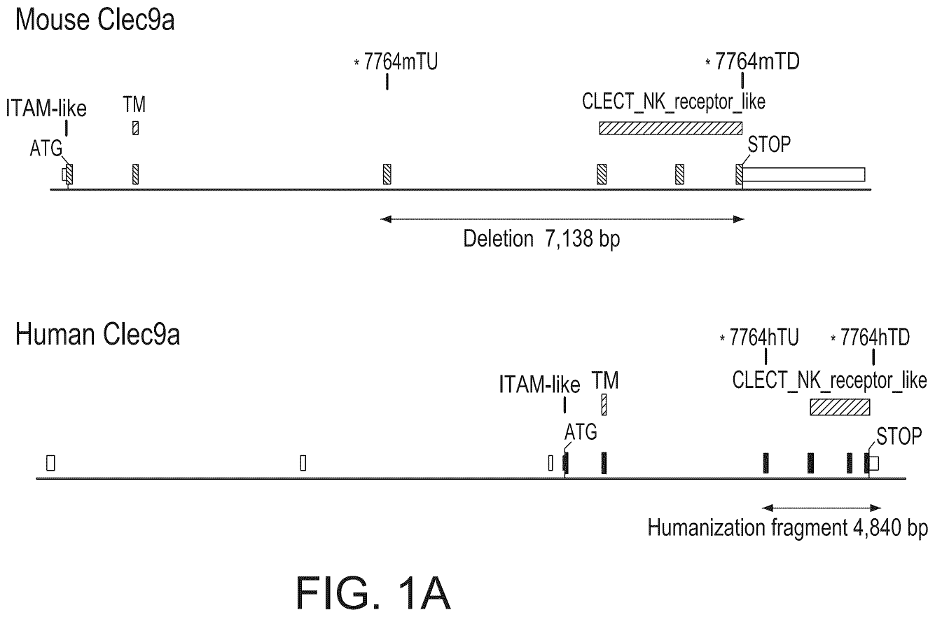

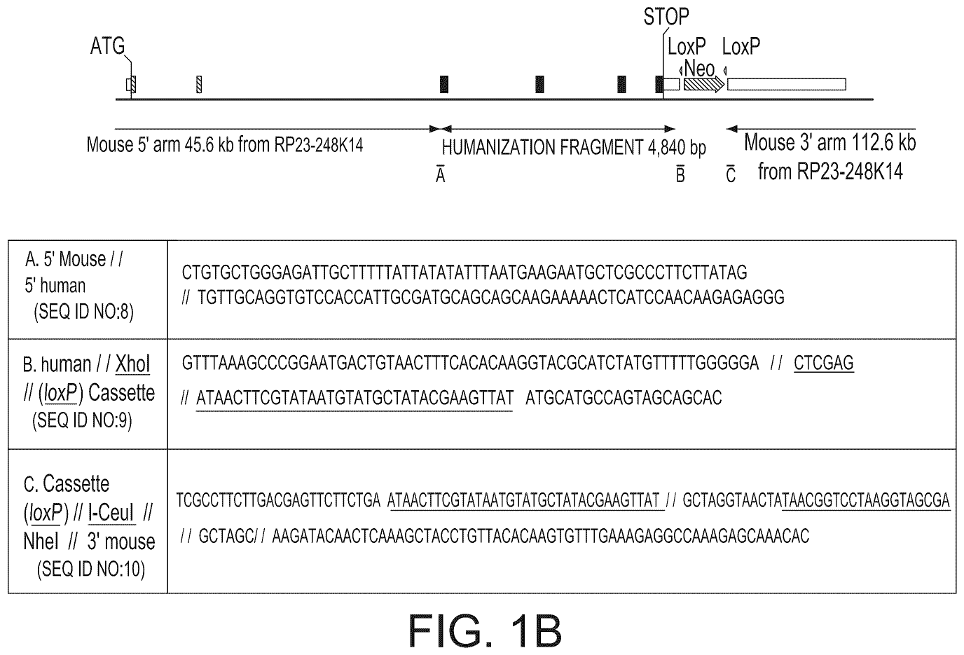

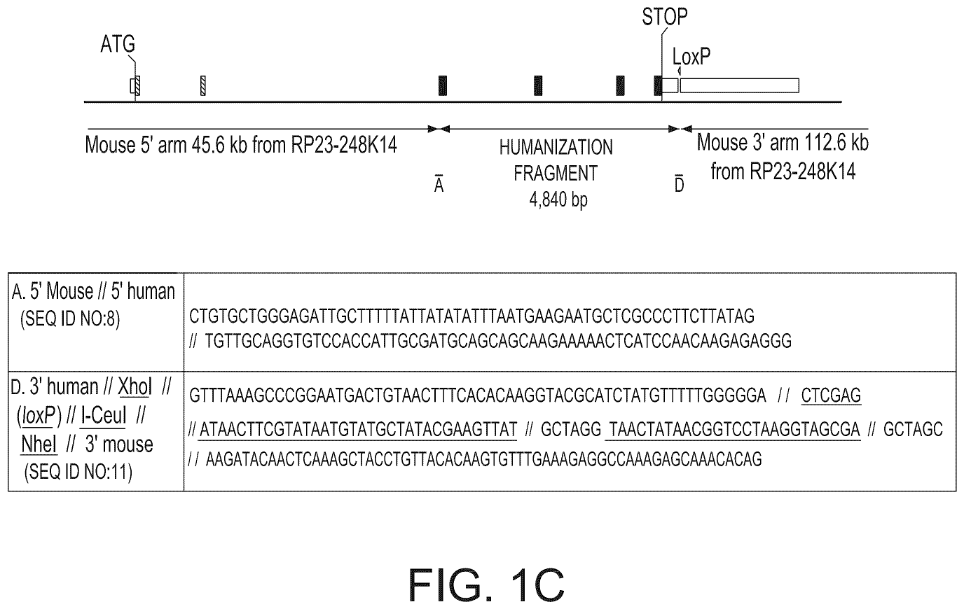

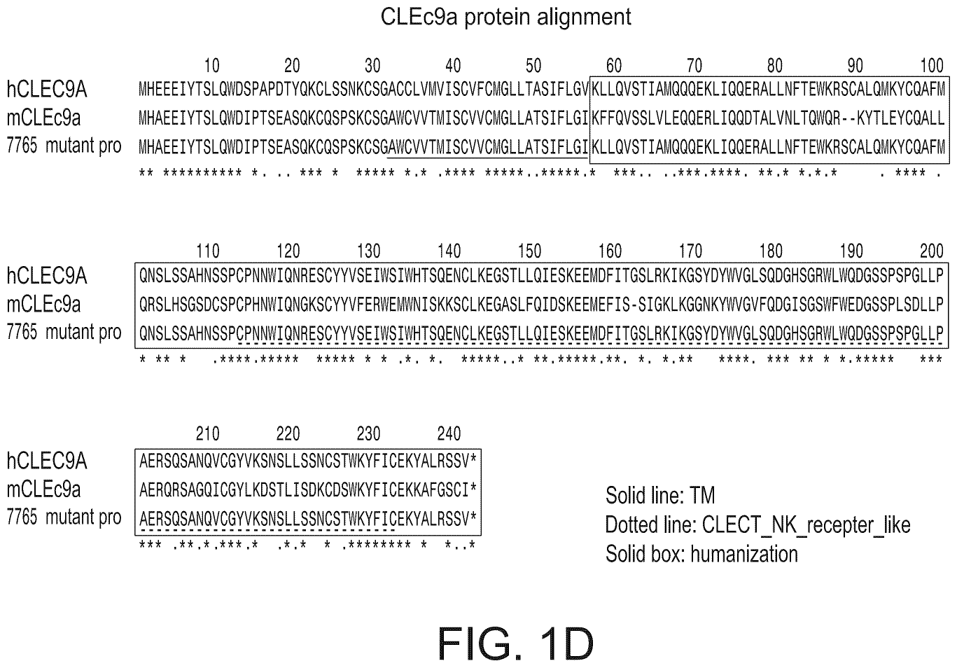

TABLE 1
Summary of Sequences

| SEQ | | |
|---|---|---|
| ID NO | Description | Features |
| 1 | Mus Musculus Clec9a, transcript | Length: 3,216 bp |
| | variant 2, mRNA, NM_172732.3 | CDS: 94-810 |
| | | Exons: 1-184; 185-265; 266-406; 407-558; |
| | | 559-677; 678-3216 |
| 2 | Mus musculus Clec9a, protein | Length: 238 aa |
| 3 | Homo sapiens CLEC9A, | Length: 1,733 bp |
| | mRNA, NM_207345.4 | CDS: 609-1334 |
| | | Exons: 1-291; 292-446; 447-550; 551-699; 700- |
| | | 780; 781-927; 928-1079; 1080-1201; 1202-1733 |
| 4 | Homo sapiens CLEC9A, protein | Length: 241 aa |
| 5 | Humanization Clec9a genomic | Length: 9,850 bp |
| | fragment | 1-100: mouse sequence |
| | | 101-4,940: human sequence (total 4,840 bp) |
| | | 4,941-9,750: XhoI-loxP-self deleting Neomycin |
| | | cassette-loxP-ICeuI-NheI (total 4,809 bp) |
| | | 9,751-9,850 bp mouse sequence |
| 6 | Humanization Clec9a genomic | Length: 5,118 bp |
| | fragment after cassette deletion | 1-100: mouse sequence |
| | | 101-4,940: human sequence (total 4,840 bp) |
| | | 4,941-5,018: XhoI-loxP-ICeuI-NheI (78 bp) |
| | | 5,019-5,118: mouse sequence |
| 7 | Humanized Clec9a protein | Length: 241 aa |
| 8 | 5′ mouse/5′ human junction | 5′mouse//5′human |
| | sequence for Clec9a | See FIG. 1 bottom panel, box A. |
| | humanization | |
| 9 | 3′ human/cassette junction | human//XhoI//(loxP) Cassette |
| | sequence for Clec9a | See FIG. 1 bottom panel, box B. |
| | humanization | |
| 10 | Cassette/3′ mouse junction | Cassette (loxP)//I-CeuI//NheI//3′ mouse |
| | sequence for Clec9a | See FIG. 1 bottom panel, box C. |
| | humanization | |
| 11 | 3′ human/loxP/3′ mouse | 3′ human//XhoI//(loxP)//I-CeuI//NheI//3′ |
| | | mouse |
| 12-23 | Primers and probes for loss of | Table 1 |
| | allele and gain of allele assays | |
| | for Clec9a humanization | |
| 24 | Rattus norvegicus Clec9a | Length: 3,253 bp |
| | mRNA, NM_001109354 | CDS: 97-822 |
| | | Exons: 1-187; 188-268; 269-415; 416-567; |
| | | 568-689; 690-3253 |
| 25 | Rattus norvegicus Clec9a protein | Length: 241 aa |

In some embodiments, rodents disclosed herein comprise a humanized Clec9a gene in the germline.

In some embodiments, a rodent disclosed herein comprises a humanized Clec9a gene in its genome that includes a nucleotide sequence of a rodent Clec9a gene (e.g., an endogenous rodent Clec9a gene) and a nucleotide sequence of a human CLEC9A gene. As used herein, “a nucleotide sequence of a gene” includes a genomic sequence, an mRNA or cDNA sequence, in full or in part of the gene. As a non-limiting example, a nucleotide sequence of a human CLEC9A gene includes a genomic sequence, an mRNA or cDNA sequence, in full or in part of the human CLEC9A gene. The nucleotide sequence of the rodent Clec9a gene and the nucleotide sequence of the human CLEC9A gene are operably linked to each other such that the humanized Clec9a gene in the rodent genome encodes a humanized Clec9a protein that maintains the protein structure of a Clec9a (comprising an ITAM-containing cytoplasmic domain, a transmembrane domain and an ectodomain) and performs functions of a Clec9a protein (e.g., recruit Syk kinase and induce proinflammatory cytokine production).

“Human CLEC9A” gene and protein, as used herein, refers to CLEC9A gene and protein of the human origin. In some embodiments, a human CLEC9A protein comprises the amino acid sequence of SEQ ID NO: 4. In some embodiments, a human CLEC9A protein comprises an amino acid sequence at least 95% identical to the amino acid sequence of SEQ ID NO: 4. In some embodiments, a human CLEC9A protein comprises an amino acid sequence at least 98% identical to the amino acid sequence of SEQ ID NO: 4. In some embodiments, a human CLEC9A protein comprises an amino acid sequence at least 99% identical to the amino acid sequence of SEQ ID NO: 4.

“Rodent Clec9a” gene and protein, as used herein, refers to Clec9a gene and protein of a rodent (e.g., mouse or rat) origin. In some embodiments, a mouse Clec9a protein comprises the amino acid sequence of SEQ ID NO: 2. In some embodiments, a mouse Clec9a protein comprises an amino acid sequence at least 95% identical to the amino acid sequence of SEQ ID NO: 2. In some embodiments, a mouse Clec9a protein comprises an amino acid sequence at least 98% identical to the amino acid sequence of SEQ ID NO: 2. In some embodiments, a mouse Clec9a protein comprises an amino acid sequence at least 99% identical to the amino acid sequence of SEQ ID NO: 2. In some embodiments, a rat Clec9a protein comprises the amino acid sequence of SEQ ID NO: 25. In some embodiments, a rat Clec9a protein comprises an amino acid sequence at least 95% identical to the amino acid sequence of SEQ ID NO: 25. In some embodiments, a rat Clec9a protein comprises an amino acid sequence at least 98% identical to the amino acid sequence of SEQ ID NO: 25. In some embodiments, a rat Clec9a protein comprises an amino acid sequence at least 99% identical to the amino acid sequence of SEQ ID NO: 25.

In some embodiments, a genetically modified rodent comprises a humanized Clec9a gene in its genome, wherein the humanized Clec9a gene encodes a humanized Clec9a protein that contains an ectodomain that is substantially identical with the ectodomain of a human CLEC9A protein. In some embodiments, an ectodomain that is substantially identical with the ectodomain of a human CLEC9A protein exhibits the same functionality (e.g., ligand binding properties) as the ectodomain of a human CLEC9A protein. An ectodomain or polypeptide that is “substantially identical with the ectodomain of a human CLEC9A protein” can be a polypeptide that is at least 95% identical in sequence with the ectodomain of a human CLEC9A protein, a polypeptide that is at least 98% identical in sequence with the ectodomain of a human CLEC9A protein, or a polypeptide that is at least 99%, identical in sequence with the ectodomain of a human CLEC9A protein. An ectodomain or polypeptide that is “substantially identical with the ectodomain of a human CLEC9A protein” can be a polypeptide that is 100% identical in sequence with the ectodomain of a human CLEC9A protein. Alternatively or additionally, an ectodomain or polypeptide that is “substantially identical with the ectodomain of a human CLEC9A protein” can be a polypeptide that differs from the ectodomain of a human CLEC9A protein by not more than 5 amino acids, a polypeptide that differs from the ectodomain of a human CLEC9A protein by not more than 4 amino acids, a polypeptide that differs from the ectodomain of a human CLEC9A protein by not more than 3 amino acids, a polypeptide that differs from the ectodomain of a human CLEC9A protein by not more than 2 amino acids, or a polypeptide that differs from the ectodomain of a human CLEC9A protein by not more than 1 amino acid. Alternatively or additionally, an ectodomain or polypeptide that is “substantially identical with the ectodomain of a human CLEC9A protein” can be a polypeptide that differs from the ectodomain of a human CLEC9A protein only at the N- or C-terminal portion of the ectodomain, e.g., by having addition, deletion and/or substitution of amino acids at the N- and/or C-terminal portion of the ectodomain (i.e., within 5-10 amino acids from the N or C terminus of the ectodomain). Alternatively or additionally, an ectodomain or polypeptide that is “substantially identical with the ectodomain of a human CLEC9A protein” can be a polypeptide that has one or more of the features delineated in above, e.g., a polypeptide that is at least 95% identical in sequence with the ectodomain of a human CLEC9A protein and differs from the ectodomain of the human CLEC9A protein only at the N- or C-terminal portion of the ectodomain by not more than 5 amino acids, or a polypeptide that is at least 98% identical in sequence with the ectodomain of a human CLEC9A protein and differs from the ectodomain of the human CLEC9A protein only at the N- or C-terminal portion of the ectodomain by not more than 3 amino acids. In some embodiments, a human CLEC9A protein comprises the amino acid sequence as set forth in SEQ ID NO: 4, and its ectodomain is composed of amino acids 57-241 of SEQ ID NO: 4. In some embodiments, a humanized Clec9a gene encodes a humanized Clec9a protein whose ectodomain is substantially identical with the ectodomain of the human CLEC9A protein as set forth in SEQ ID NO: 4, i.e., substantially identical with amino acids 57-241 of SEQ ID NO: 4. For example, the humanized Clec9a gene encodes a humanized Clec9a protein having an ectodomain that comprises amino acids 57-241, 58-241, 59-241, 60-241, 57-240, 57-239, 57-238, or 57-237 of SEQ ID NO: 4. In some embodiments, the humanized Clec9a gene encodes a humanized Clec9a protein having an ectodomain that comprises amino acids 57-241 of SEQ ID NO: 4. In some embodiments, the humanized Clec9a gene encodes a humanized Clec9a protein having an ectodomain that comprises amino acids 58-241 of SEQ ID NO: 4. In some embodiments, the humanized Clec9a gene encodes a humanized Clec9a protein having an ectodomain that comprises amino acids 59-241 of SEQ ID NO: 4. In some embodiments, the humanized Clec9a gene encodes a humanized Clec9a protein having an ectodomain that comprises amino acids 57-240 of SEQ ID NO: 4. In some embodiments, the humanized Clec9a gene encodes a humanized Clec9a protein having an ectodomain that comprises amino acids 57-239 of SEQ ID NO: 4.

In some embodiments, the humanized Clec9a gene encodes a humanized Clec9a protein that contains a cytoplasmic-transmembrane sequence (i.e., a sequence that includes both the transmembrane domain and the cytoplasmic domain) that is substantially identical with the cytoplasmic-transmembrane sequence of a rodent Clec9a protein, e.g., an endogenous rodent Clec9a protein. In some embodiments, a cytoplasmic-transmembrane sequence that is substantially identical with the cytoplasmic-transmembrane sequence of an endogenous rodent Clec9a protein exhibits the same functionality (e.g., signal transduction and/or interaction with intracellular molecules) as the cytoplasmic-transmembrane sequence of a rodent Clec9a protein such as an endogenous rodent Clec9a protein. A cytoplasmic-transmembrane sequence or polypeptide that is “substantially identical with the cytoplasmic-transmembrane sequence of a rodent Clec9a protein” can be a polypeptide that is at least 95% identical in sequence with the cytoplasmic-transmembrane sequence of a rodent Clec9a protein, or a polypeptide that is at least 98% identical in sequence with the cytoplasmic-transmembrane sequence of a rodent Clec9a protein. A cytoplasmic-transmembrane sequence or polypeptide that is “substantially identical with the cytoplasmic-transmembrane sequence of a rodent Clec9a protein” can be a polypeptide that is identical with the cytoplasmic-transmembrane sequence of a rodent Clec9a protein.

Alternatively or additionally, a cytoplasmic-transmembrane sequence or polypeptide that is “substantially identical with the cytoplasmic-transmembrane sequence of a rodent Clec9a protein” can be a polypeptide that differs from the cytoplasmic-transmembrane sequence of a rodent Clec9a protein by not more than 3 amino acids, a polypeptide that differs from the cytoplasmic-transmembrane sequence of a rodent Clec9a protein by not more than 2 amino acids, or a polypeptide that differs from the cytoplasmic-transmembrane sequence of a rodent Clec9a protein by not more than 1 amino acid. Alternatively or additionally, a cytoplasmic-transmembrane sequence or polypeptide that is “substantially identical with the cytoplasmic-transmembrane sequence of a rodent Clec9a protein” can be a polypeptide that differs from the cytoplasmic-transmembrane sequence of a rodent Clec9a protein only at the N- or C-terminus, e.g., by having addition, deletion or substitution of amino acids at the N- or C-terminal portion of the transmembrane-cytoplasmic sequence. Alternatively or additionally, a cytoplasmic-transmembrane sequence or polypeptide that is “substantially identical with the cytoplasmic-transmembrane sequence of a rodent Clec9a protein” can be a polypeptide having one or more features delineated in above, e.g., a polypeptide that is at least 95% identical in sequence with the cytoplasmic-transmembrane sequence of a rodent Clec9a protein, and differs from the cytoplasmic-transmembrane sequence of a rodent Clec9a protein only at the N- or C-terminus by not more than 3 amino acids; or a polypeptide that is at least 95% identical in sequence with the cytoplasmic-transmembrane sequence of a rodent Clec9a protein, and differs from the cytoplasmic-transmembrane sequence of a rodent Clec9a protein only at the N- or C-terminus by not more than 2 amino acids. By “the N- or C-terminal portion of the cytoplasmic-transmembrane sequence” is meant within 3-5 amino acids from the N-terminus of the cytoplasmic domain or from the C-terminus of the transmembrane domain. In some embodiments, a humanized Clec9a protein contains a cytoplasmic-transmembrane sequence that is substantially identical with the cytoplasmic-transmembrane sequence of a mouse Clec9a protein (such as an endogenous mouse Clec9a protein). In some embodiments, a humanized Clec9a protein contains a cytoplasmic-transmembrane sequence that is substantially identical with the cytoplasmic-transmembrane sequence of a rat Clec9a protein (such as an endogenous rat Clec9a protein).

In some embodiments, the humanized Clec9a gene in the genome of a genetically modified rodent includes a nucleotide sequence of a human CLEC9A gene (“a human CLEC9A nucleotide sequence”) and a nucleotide sequence of a rodent Clec9a gene (“a rodent Clec9a nucleotide sequence”, such as an endogenous rodent Clec9a nucleotide sequence), wherein the human CLEC9A nucleotide sequence encodes at least a substantial portion of the ectodomain of a human CLEC9A protein. Examples of a substantial portion of the ectodomain of a human CLEC9A can include amino acids 57-241, 57-240, 57-239, 58-241, or 59-241 of SEQ ID NO: 4. In some embodiments, a substantial portion of the ectodomain comprises amino acids 57-241 of SEQ ID NO: 4. In some embodiments, a substantial portion of the ectodomain comprises amino acids 57-240 of SEQ ID NO: 4. In some embodiments, a substantial portion of the ectodomain comprises amino acids 57-239 of SEQ ID NO: 4. In some embodiments, a substantial portion of the ectodomain comprises amino acids 58-241 of SEQ ID NO: 4. In some embodiments, a substantial portion of the ectodomain comprises amino acids 59-241 of SEQ ID NO: 4. In some embodiments, the human CLEC9A nucleotide sequence is a cDNA sequence. In some embodiments, the human CLEC9A nucleotide sequence in a humanized Clec9a gene encodes the ectodomain of a human CLEC9A protein (e.g., a human CLEC9A protein as defined in SEQ ID NO: 4). In some embodiments, the human CLEC9A nucleotide sequence is a genomic fragment of a human CLEC9A gene. In some embodiments, the human CLEC9A nucleotide sequence is a genomic fragment of a human CLEC9A gene comprising exon 3 through the Stop codon in exon 6. In some embodiments, the human CLEC9A nucleotide sequence is a genomic fragment of a human CLEC9A gene comprising exon 3 through exon 6, i.e., through the 3′ untranslated region in exon 6.

In some embodiments, the humanized Clec9a gene in the genome of a genetically modified rodent includes a rodent Clec9a nucleotide sequence and a human CLEC9A nucleotide sequence, wherein the rodent Clec9a nucleotide sequence encodes a polypeptide substantially identical to the cytoplasmic-transmembrane sequence of a rodent Clec9a protein (e.g., an endogenous rodent Clec9a protein). In some embodiments, the rodent Clec9a nucleotide sequence present in a humanized Clec9a gene encodes the cytoplasmic-transmembrane sequence of an endogenous rodent Clec9a protein. In some embodiments, the rodent Clec9a nucleotide sequence present in a humanized Clec9a gene comprises exon 1 (in full or in part, e.g., the coding portion) and exon 2 of a rodent (e.g., endogenous rodent) Clec9a gene. In some embodiments, the rodent Clec9a nucleotide sequence present in a humanized Clec9a gene is a mouse Clec9a nucleotide sequence; and in some such embodiments, the mouse Clec9a nucleotide sequence comprises the coding portion exon 1 (and optionally also including the 5′ UTR of exon 1) and exon 2 of a mouse Clec9a gene (e.g., an endogenous mouse Clec9a gene). In some embodiments, the rodent Clec9a nucleotide sequence present in a humanized Clec9a gene also comprises the 3′ UTR of a rodent Clec9a gene. In some embodiments, the 3′ UTR of a rodent Clec9a gene is placed downstream of the 3′ UTR of a human CLEC9A gene.

In some embodiments, the humanized Clec9a gene is operably linked to rodent Clec9a 5′ regulatory sequences such as endogenous rodent Clec9a regulatory sequences, e.g., a 5′ transcriptional regulatory sequence(s) such as promoter and/or enhancers, such that expression of the humanized Clec9a gene is under control of the rodent Clec9a 5′ regulatory sequence(s).

In some embodiments, the humanized Clec9a gene is at an endogenous rodent Clec9a locus. In some embodiments, the humanized Clec9a gene is at a locus other than an endogenous rodent Clec9a locus; e.g., as a result of random integration. In some embodiments, the humanized Clec9a gene is at a ROSA26 locus (which locus is as described by Zambrowicz et al., 1997, PNAS USA 94:3789-3794, which is incorporated herein by reference). In some embodiments where the humanized Clec9a gene is at a locus other than an endogenous rodent Clec9a locus, the rodents are incapable of expressing a rodent Clec9a protein, e.g., as a result of inactivation (e.g., deletion in full or in part) of the endogenous rodent Clec9a gene.

In some embodiments where a humanized Clec9a gene is at an endogenous rodent Clec9a locus, the humanized Clec9a gene results from a replacement of a nucleotide sequence of an endogenous rodent Clec9a gene at the endogenous rodent Clec9a locus with a nucleotide sequence of a human CLEC9A gene.

In some embodiments, the nucleotide sequence of an endogenous rodent Clec9a gene at an endogenous rodent Clec9a locus that is being replaced is a genomic fragment of an endogenous rodent Clec9a gene that encodes at least a substantial portion of the ectodomain of the rodent Clec9a protein. In some embodiments, the rodent is a mouse, and the mouse Clec9a genomic fragment being replaced encodes at least a substantial portion of the ectodomain of the endogenous mouse Clec9a protein. For example, the ectodomain of a mouse Clec9a of SEQ ID NO: 2 is defined by amino acids 57-238, examples of a substantial portion of the ectodomain can include amino acids 57-238, 57-237, 57-236, 58-238, or 59-238 of SEQ ID NO: 2. In some embodiments, a substantial portion of the ectodomain of a mouse Clec9a protein comprises amino acids 57-238 of SEQ ID: 2. In some embodiments, a substantial portion of the ectodomain of a mouse Clec9a protein comprises amino acids 57-237 of SEQ ID: 2. In some embodiments, a substantial portion of the ectodomain of a mouse Clec9a protein comprises amino acids 57-236 of SEQ ID: 2. In some embodiments, a substantial portion of the ectodomain of a mouse Clec9a protein comprises amino acids 58-238 of SEQ ID: 2. In some embodiments, a substantial portion of the ectodomain of a mouse Clec9a protein comprises amino acids 59-238 of SEQ ID: 2. In some embodiments, the mouse Clec9a genomic fragment being replaced comprises exon 3 through the Stop codon in exon 6.

In some embodiments, the nucleotide sequence of a human CLEC9A gene that replaces a genomic fragment of a rodent Clec9a gene at an endogenous rodent Clec9a locus is a cDNA sequence. In some embodiments, the human CLEC9A nucleotide sequence that replaces a genomic fragment of a rodent Clec9a gene at an endogenous rodent Clec9a locus is a genomic fragment of a human CLEC9A gene. In some embodiments, a genomic fragment of a human CLEC9A gene that replaces a genomic fragment of a rodent Clec9a gene at an endogenous rodent Clec9a locus includes exons, in full or in part, of a human CLEC9A gene, that encode at least a substantial portion of the ectodomain of the human CLEC9A protein. Examples of a substantial portion of the ectodomain of a human CLEC9A have been described above, e.g., amino acids 57-241, 57-240, 57-239, 58-241, or 59-241 of SEQ ID NO: 4. In some embodiments, a substantial portion of the ectodomain of a human CLEC9A comprises amino acids 57-241 of SEQ ID NO: 4. In some embodiments, a substantial portion of the ectodomain of a human CLEC9A comprises amino acids 57-240 of SEQ ID NO: 4. In some embodiments, a substantial portion of the ectodomain of a human CLEC9A comprises amino acids 57-239 of SEQ ID NO: 4. In some embodiments, a substantial portion of the ectodomain of a human CLEC9A comprises amino acids 58-241 of SEQ ID NO: 4. In some embodiments, a substantial portion of the ectodomain of a human CLEC9A comprises amino acids 59-241 of SEQ ID NO: 4. In some embodiments, the human genomic fragment comprises human CLEC9A exon 3 through the Stop codon in exon 6. In some embodiments, the human genomic fragment comprises human CLEC9A exon 3 through the 3′ end of exon 6 (i.e., including the 3′ UTR of human CLEC9A).

In some embodiments, the human CLEC9A nucleotide sequence inserted into an endogenous rodent Clec9a locus is operably linked to a genomic sequence of a rodent Clec9a gene that encodes a polypeptide substantially identical to the cytoplasmic-transmembrane sequence of a rodent Clec9a protein (such as an endogenous rodent, e.g., mouse or rat, Clec9a protein). In some embodiments, the genomic sequence of a rodent Clec9a gene comprises exon 1 and exon 2 of a rodent Clec9a gene (e.g., an endogenous mouse or rat Clec9a gene). In some embodiments, the genomic sequence of a rodent Clec9a gene also comprises the 3′ UTR of a rodent Clec9a gene.

In some embodiments, the rodent is a mouse, and a genomic fragment of an endogenous mouse Clec9a gene at an endogenous mouse Clec9a locus comprising exon 3 through the Stop codon in exon 6 of the mouse Clec9a gene (encoding the mouse Clec9a ectodomain) has been replaced with a genomic fragment of a human CLEC9A gene comprising exon 3 through the Stop codon in exon 6 coding for the human CLEC9A ectodomain. In some embodiments, a humanized Clec9a gene is formed at the endogenous mouse Clec9a locus and comprises exons 1-2 of a mouse Clec9a gene, exon 3 through exon 6 of a human CLEC9A gene, optionally followed by the 3′ UTR of the mouse Clec9a gene.

In some embodiments, a rodent provided herein is heterozygous for a humanized Clec9a gene in its genome. In some embodiments, a rodent provided herein is homozygous for a humanized Clec9a gene in its genome.

In some embodiments, a humanized Clec9a gene results in an expression of the encoded humanized Clec9a protein in a rodent. In some embodiments, a humanized Clec9a protein is expressed in cells and tissues in which a counterpart rodent Clec9a protein in a control rodent (e.g., a rodent without the humanized Clec9a gene) is typically expressed, for example, on dendritic cells.

In some embodiments, rodents disclosed herein are incapable of expressing a rodent Clec9a protein, e.g., as a result of inactivation (e.g., deletion in full or in part) or replacement (in full or in part) of the endogenous rodent Clec9a gene.

### Rodent Species and Strains

In some embodiments, rodents of this disclosure include, as non-limiting examples, a mouse, a rat, and a hamster. In some embodiments, a rodent is selected from the superfamily Muroidea. In some embodiments, a rodent of this disclosure is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, a rodent of this disclosure is selected from a true mouse or rat (family Muridae), a gerbil, a spiny mouse, and a crested rat. In some embodiments, a mouse of this disclosure is from a member of the family Muridae.

In some embodiments, a rodent is a mouse. In some embodiments, the rodent is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, a rodent is a mouse of a 129 strain selected from the group consisting of a strain that is 129P1, 129P2, 129P3, 129X1, 129S1 (e.g., 129S1/SV, 129S1/SvIm), 129S2, 129S4, 12955, 129S9/SvEvH, 129/SvJae, 129S6 (129/SvEvTac), 129S7, 12958, 129T1, 129T2 (see, e.g., Festing et al., 1999, Mammalian Genome 10:836; Auerbach et al., 2000, Biotechniques 29(5):1024-1028, 1030, 1032; both incorporated herein by reference in their entireties). In some embodiments, a rodent is a mouse that is a mix of a 129 strain and a C57BL/6 strain. In some embodiments, a rodent is a mouse that is a mix of aforementioned 129 strains, or a mix of aforementioned BL/6 strains. In some embodiments, a rodent is a mouse of a BALB strain, e.g., BALB/c strain. In some embodiments, a rodent is a mouse that is a mix of a BALB strain and another aforementioned strain.

In some embodiments, a rodent is a rat. In some certain embodiments, a rat is selected from a Wistar rat, an LEA strain, a Sprague Dawley strain, a Fischer strain, F344, F6, and Dark Agouti. In some embodiments, a rat strain as described herein is a mix of two or more strains selected from the group consisting of Wistar, LEA, Sprague Dawley, Fischer, F344, F6, and Dark Agouti.

### Tissues and Cells of Genetically Modified Rodents

In some embodiments, disclosed herein is an isolated rodent cell or tissue whose genome comprises a humanized Clec9a gene.

In some embodiments, a tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof.

In some embodiments, a cell is selected from a dendritic cell or a monocyte.

In some embodiments, an isolated rodent cell is a rodent embryonic stem cell. In some embodiments, an isolated rodent cell is a rodent egg, or a rodent sperm.

### Compositions and Methods for Making Humanized Rodents

Disclosed herein is a targeting vector (or nucleic acid construct) comprising a human CLEC9A nucleotide sequence, desired to be integrated into a rodent locus to form a humanized Clec9a gene as described herein.

In some embodiments, a targeting vector comprises a human CLEC9A nucleotide sequence which encodes at least a substantial portion of the ectodomain of a human CLEC9A protein as described hereinabove. In some embodiments, the human CLEC9A nucleotide sequence encodes a polypeptide comprising amino acids 57-241 of SEQ ID NO: 4. In some embodiments, the human CLEC9A nucleotide sequence comprises exon 3 through the Stop codon in exon 6 encoding the ectodomain of a human CLEC9A protein.

The targeting vector also includes 5′ and 3′ rodent sequences flanking the human nucleotide sequence to be integrated, also known as 5′ and 3′ homology arms, that mediate homologous recombination and integration of the human nucleotide sequence into the target rodent locus (e.g., an endogenous rodent Clec9a locus), so as to form a humanized gene as described herein above. Typically, the 5′ and 3′ flanking rodent sequences in a targeting vector are identical or substantially identical (e.g., at least 98% or at least 99% identical) to the nucleotide sequences that flank the corresponding rodent nucleotide sequence at the target rodent locus that is to be replaced by the human nucleotide sequence. In some embodiments, a targeting vector comprises a humanized gene as described herein above. In some embodiments, a targeting vector comprises a humanized Clec9a gene comprising a human CLEC9A nucleotide sequence and a rodent Clec9a nucleotide sequence, as described herein above. In some embodiments, a targeting vector comprises a humanized Clec9a gene, wherein the humanized Clec9a gene comprises exons 1-2 of a rodent Clec9a gene and exons 3-6 of a human CLEC9A gene, and optionally the humanized Clec9a gene is flanked by 5′ and 3′ rodent homology arms.

In some embodiments, a targeting vector comprises a selection marker gene. The selection marker gene can be inserted in an intron of the human genomic sequence to be integrated. In some embodiments, a selection marker gene is provided as a self-deleting cassette which can be deleted after a successful integration of the human nucleotide sequence.

In an exemplary embodiment, a targeting vector is generated from a bacterial artificial chromosome (BAC) clone carrying a rodent Clec9a genomic DNA using bacterial homologous recombination and VELOCIGENE@ technology (see, e.g., U.S. Pat. No. 6,586,251 and Valenzuela et al. (2003) Nature Biotech. 21(6):652-659, incorporated herein by reference in their entireties). As a result of bacterial homologous recombination, a rodent genomic sequence is deleted from the BAC clone, and a human nucleotide sequence is inserted, resulting in a modified BAC clone carrying the human nucleotide sequence, flanked with 5′ and 3′ rodent homology arms. In some embodiments, the human nucleotide sequence can be a cDNA sequence or a human genomic DNA. The modified BAC clone, once linearized, can be introduced into rodent embryonic stem (ES) cells.

In some embodiments, the present invention provides use of a targeting vector as described herein to make a modified rodent embryonic stem (ES) cell. A targeting vector can be introduced into a rodent ES cell by, e.g., electroporation. Both mouse ES cells and rat ES cells have been described in the art. See, e.g., U.S. Pat. Nos. 7,576,259, 7,659,442, 7,294,754, and US 2008-0078000 A1 (all of which are incorporated herein by reference in their entireties) that describe mouse ES cells and the VELOCIMOUSE® method for making a genetically modified mouse; US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties) that describe rat ES cells and methods for making a genetically modified rat, which can be used to make a modified rodent embryo, which in turn can be used to make a rodent animal.

In some embodiments, ES cells having a desirable human nucleotide sequence (e.g., a human CLEC9A nucleotide sequence) integrated in the genome can be selected. In some embodiments, ES cells are selected based on loss of rodent allele and/or gain of human allele assays. In some embodiments, selected ES cells are then used as donor ES cells for injection into a pre-morula stage embryo (e.g., 8-cell stage embryo) by using the VELOCIMOUSE® method (see, e.g., U.S. Pat. Nos. 7,576,259, 7,659,442, 7,294,754, and US 2008-0078000 A1, all of which are incorporated by reference in their entireties), or methods described in US 2014/0235933 A1 and US 2014/0310828 A1, which are both incorporated by reference in their entireties. In some embodiments, an embryo comprising the donor ES cells is incubated and implanted into a surrogate mother to produce an FO rodent. Rodent pups bearing a human nucleotide sequence can be identified by genotyping of DNA isolated from tail snips using loss of rodent allele and/or gain of human allele assays.

In some embodiments, rodents heterozygous for a humanized gene can be crossed to generate homozygous rodents.

A humanized rodent as described herein (i.e., a rodent comprising a humanized Clec9a gene) can be bred or crossed with another rodent. Accordingly, methods of breeding as well as progenies obtained from such breeding are also embodiments of this disclosure.

In some embodiments, a method is provided which comprises breeding a first rodent as described hereinabove, e.g., a rodent whose genome comprises a humanized Clec9a gene, with a second rodent, resulting in a progeny rodent whose genome comprises the humanized Clec9a, gene. The progeny may possess other desirable phenotypes or genetic modifications inherited from the second rodent used in the breeding. In some embodiments, the progeny rodent is heterozygous for the humanized gene or genes from the first rodent. In some embodiments, the progeny rodent is homozygous for the humanized gene(s) from the first rodent.

In some embodiments, a progeny rodent is provided whose genome comprises a humanized Clec9a gene, wherein the progeny rodent is produced by a method comprising breeding a first rodent whose genome comprises the humanized Clec9a gene, with a second rodent. In some embodiments, the progeny rodent is heterozygous for the humanized Clec9a gene from the first rodent. In some embodiments, the progeny rodent is homozygous for the humanized Clec9a gene from the first rodent.

In some embodiments, disclosed herein is an in vitro method for generating a genetically modified rodent cell, comprising introducing into a rodent cell a targeting vector comprising a human CLEC9A nucleic sequence that encodes at least a substantial portion of the ectodomain of a human CLEC9A protein, flanked by rodent homology arms that mediate integration of the human CLEC9A nucleotide sequence into an endogenous rodent Clec9a locus, which results in replacement of a rodent Clec9a genomic DNA with the human CLEC9A nucleic acid sequence to form a humanized Clec9a gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

### Methods Employing the Humanized Rodents

Rodents disclosed herein provide a useful in vivo system and source of biological materials for identifying and testing compounds for their potential to treat human diseases, including immune related indications, i.e., conditions that may involve or result from dysregulated immune functions such as autoimmunity, infectious disease, and cancer, among others in which CLEC9A may play a role.

In some embodiments, genetically modified rodent animals disclosed herein are used to evaluate agents that target human CLEC9A. In some embodiments, the agent is an antibody that specifically binds to human CLEC9A. A candidate agent such as an anti-human CLEC9A antibody can be administered to a rodent disclosed herein at various doses (e.g., 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/mg, 7.5 mg/kg, 10 mg/kg, 15 mg/kg, 20 mg/kg, 25 mg/kg or more). The agents may be dosed via any desired route of administration (e.g., subcutaneously, intravenously, intramuscular, intraperitoneal, etc.).

In some embodiments, genetically modified rodent animals disclosed herein are used for assessing pharmacokinetic properties of a candidate agent such as an anti-human CLEC9A antibody. The agent is administered to a genetically modified rodent animal. Blood is isolated from the animal at various time points (e.g., 0 hr, 6 hr, 1 day, 2 days, 3 days, 4 days, 5 days, 6 days, 7 days, 8 days, 9 days, 10 days, 11 days, or up to 30 or more days). Various assays may be performed to determine the pharmacokinetic properties which can include, but are not limited to, how an animal processes the drug into various metabolites (or detection of the presence or absence of one or more drug metabolites, including, toxic metabolites), drug half-life, circulating levels of drug after administration (e.g., serum concentration of drug), anti-drug response (e.g., anti-drug antibodies), drug absorption and distribution, route of administration, routes of excretion and/or clearance of the drug.

In some embodiments, genetically modified rodent animals disclosed herein are used to evaluate an agent that target human CLEC9A (e.g., an antibody) in order to determine whether the agent has an effect on the rodent animals, e.g., whether the agent triggers dendritic cell dependent activation of immune cells (such as T cells). Such evaluation can be accomplished by administering the agent to a rodent animal disclosed herein and assessing the immune cells in the rodent to determine whether there is activation of the immune cells, e.g., proliferation of T cells, and/or production of cytokines. Comparison can be made to a control rodent animal that is not administered with the agent, or is administered with a control agent (e.g., an isotype antibody). In some embodiments, a rodent animal disclosed herein, e.g., a mouse or rat expressing a humanized Clec9a protein on dendritic cells, is administered with an antibody-peptide fusion, wherein the antibody binds to the human CLEC9A ectodomain, and wherein the peptide comprises one or more antigens recognized by major histocompatibility complex (MHC) molecules on dendritic cells. In some embodiments, the peptide comprises an antigen recognized by MHC class I molecules (e.g., amino acids 257-264 of ovalbumin, also known as the OTI peptide), and an antigen recognized by MHC class II molecules (e.g., amino acids 323-339 of ovalbumin, also known as the OTII peptide). The rodent animal is then examined to determine whether the exposure to the antibody-peptide fusion induces a T-cell response (e.g., T cell proliferation). In some embodiments, the rodent animal is examined to measure whether there is a proliferation of CD4+ T cells. In some embodiments, the rodent animal is examined to measure whether there is a proliferation of CD8+ T cells. Methods and assays for evaluating a T cell response in an animal are well known in the art and are also illustrated in the Examples section hereinbelow. Comparison can be made to a control rodent animal that is not administered with the antibody-peptide fusion or is administered with an isotype antibody-peptide fusion, with an isotype antibody, or with the peptide alone. Any observed T cell response and the extent of the response may be correlated with the efficacy of the antibody being tested.

The present description is further illustrated by the following examples, which should not be construed as limiting in any way. The contents of all cited references (including literature references, issued patents, and published patent applications as cited throughout this application) are hereby expressly incorporated by reference in their entireties.

### Example 1. Humanization of an Endogenous Mouse Clec9a Gene

A targeting vector for humanization of an endogenous Clec9a gene was constructed using bacterial artificial chromosome (BAC) clones and VELOCIGENE® technology (see, e.g., U.S. Pat. No. 6,586,251 and Valenzuela et al. (2003) High-throughput engineering of the mouse genome coupled with high-resolution expression analysis, Nature Biotechnology 21 (6):652-659; all of the contents of which are incorporated herein by reference).

A BAC clone RP23-248K14 containing a mouse Clec9a gene was used and modified as follows. Briefly, a DNA fragment was generated to include a mouse 5′ homology nucleotide sequence (100 bp), a human CLEC9A genomic DNA of 4,840 bp (containing exons 3, 4, 5, 6 and their respective introns 3, 4, 5 (including the full human 3′ UTR) of a human CLEC9A gene), a self-deleting Neomycin cassette of 4,809 bp, and a 3′ mouse homology sequence (100 bp). This DNA fragment was used to modify BAC clone RP23-248K14 through homologous recombination in bacterial cells. As result, a mouse Clec9a genomic fragment of 7,138 bp (encoding the ectodomain of the mouse Clec9a protein) in the BAC clone was replaced by the human CLEC9A genomic fragment of 4,840 bp, followed by a self-deleting Neomycin cassette of 4,809 bp. Specifically, the mouse Clec9a genomic fragment that was replaced included the entire exon 3 through the stop codon of the last coding exon (exon 6) of mouse Clec9a gene (FIGS. 1A-1B). The human CLEC9A genomic fragment that was inserted included exons 3, 4, 5 with their respective introns through the stop codon of the last coding exon (exon 6), including the full 3′ UTR of human CLEC9A (FIGS. 1A-1B). The resulting modified BAC clone included, from 5′ to 3′, (i) a 5′ mouse homology arm containing about 45.6 kb of mouse genomic DNA including a mouse Clec9a 5′ UTR, mouse Clec9a exons 1 and 2; (ii) a human CLEC9A genomic fragment of about 4,840 bp including exons 3, 4, 5, and 6 (including the 3′ UTR of human CLEC9A); (iii) a self-deleting Neomycin cassette of about 4,809 bp, followed by (iv) a 3′ mouse homology arm of 112.6 kb containing the mouse Clec9a 3′ UTR and the remaining mouse genomic DNA in the original BAC clone (FIGS. 1A-1B). The junction sequences are also set forth at the bottom or FIG. 1B. The part of the modified BAC clone containing the human CLEC9A genomic fragment and the self-deleting Neomycin cassette, as well as the upstream and downstream insertion junctions, is set forth in SEQ ID NO: 5. The amino acid sequence of the protein encoded by the humanized Clec9a gene is set forth in SEQ ID NO: 7 and FIG. 1D. An alignment of this humanized Clec9a protein (“7765 mutant protein”), a mouse Clec9a protein (SEQ ID NO: 2), and a human CLEC9A protein (SEQ ID NO: 4), is provided in FIG. 1D.

The modified BAC clone containing the humanized Clec9a gene, as described above, was used to electroporate mouse embryonic stem (ES) cells to create modified ES cells comprising a humanized Clec9a gene. Positively targeted ES cells containing a humanized Clec9a gene were identified by an assay (Valenzuela et al., supra) that detected the presence of the human CLEC9A sequences (e.g., exons 3-6 of human CLEC9A) and confirmed the loss and/or retention of mouse Clec9a sequence (e.g., loss of exons 3-6 of mouse Clec9a). Table 1 sets forth the primers and probes that were used to confirm humanization of an endogenous Clec9a gene as described above (FIGS. 1A-1B). A correctly targeted ES cell clone was selected as donor cell and injected into a pre-morula stage embryo (e.g., 8-cell stage embryo) by using the VELOCIMOUSE® method (see, e.g., Poueymirou et al., 2007, Nature Biotech. 25(1):91-99, U.S. Pat. Nos. 7,576,259, 7,659,442, 7,294,754, and US 2008-0078000 A1, all of which are incorporated herein by reference in their entireties), or methods described in US 2014/0235933 A1 and US 2014/0310828 A1 (both of which incorporated herein by reference in their entireties). The embryo comprising the donor ES cells was incubated until blastocyst stage and then implanted into a surrogate mother to produce an FO rodent fully derived from the donor ES cells. Mice bearing a humanized Clec9a allele were confirmed and identified by genotyping of DNA isolated from tails snips using a modification of allele assay (Valenzuela et al., supra) that detects the presence of the human CLEC9A gene sequences. The neomycin selection cassette was removed by crossing the progeny generated from the ES clone with a deletor rodent strain that expresses a Cre recombinase. The humanized Clec9a locus after the deletion of the cassette is depicted in FIG. 1C, with the junction sequences shown at the bottom of FIG. 1C. Mice heterozygous for the humanized Clec9a locus were crossed to obtain homozygotes.

TABLE 2

| NAME | Primer | Sequence (5′-3′) | SEQ ID NO |
|---|---|---|---|
| 7764mTU | Forward | GGAGCAGCAGGAAAGACTCA | (SEQ ID NO: 12) |
| | Probe (BHQ) | TCCAACAGGACACAGCATTGGTGA | (SEQ ID NO: 13) |
| | Reverse | GCTTGGCAGTATTCCAGTGTGTA | (SEQ ID NO: 14) |
| 7764mTD | Forward | GGTTGCCAGCAGAAAGACA | (SEQ ID NO: 15) |
| | Probe (BHQ) | ATCAGCCGGCCAGATCTGTGGA | (SEQ ID NO: 16) |
| | Reverse | TCCAGCTATCGCACTTATCTGA | (SEQ ID NO: 17) |
| 7764hTU | Forward | ATTGCGATGCAGCAGCAAGAAA | (SEQ ID NO: 18) |
| | Probe (BHQ) | CATCCAACAAGAGAGGGCACTGC | (SEQ ID NO: 19) |
| | Reverse | GGCACAGCTTCTCTTCCATTC | (SEQ ID NO: 20) |
| 7764hTD | Forward | GCAACCTGGGACTCAATAATACAC | (SEQ ID NO: 21) |
| | Probe (BHQ) | TGGGAATATTCTTCCACACCGTCCA | (SEQ ID NO: 22) |
| | Reverse | ACTCTTGCAATGTGAACAACATCA | (SEQ ID NO: 23) |

### Example 2. Characterization of the Genetically Modified Mice Containing a Humanized Clec9a Gene

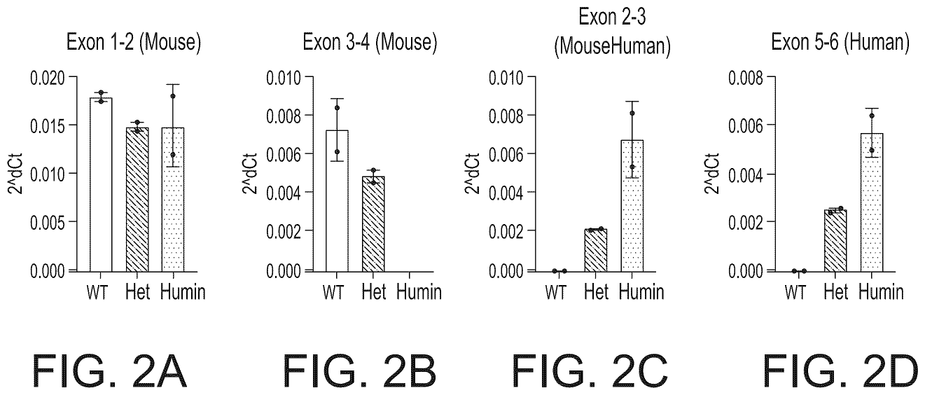

To determine whether the genetically modified mice described in Example 1 expressed humanized Clec9a, mRNA expression was examined. RNA was extracted from splenocytes from wild-type (WT) mice (without humanization of the endogenous Clec9a gene), mice heterozygous for the humanized Clec9a gene (Het), or mice homozygous for the humanized Clec9a gene (HumIn) and subjected to RT-PCR using probes that detect mouse or human Clec9a. This line of experimentation showed that the WT and Het mice expressed mouse Clec9a (FIGS. 2A and 2B). These results also showed that the Het and HumIn mice expressed humanized Clec9a (FIGS. 2C-2D).

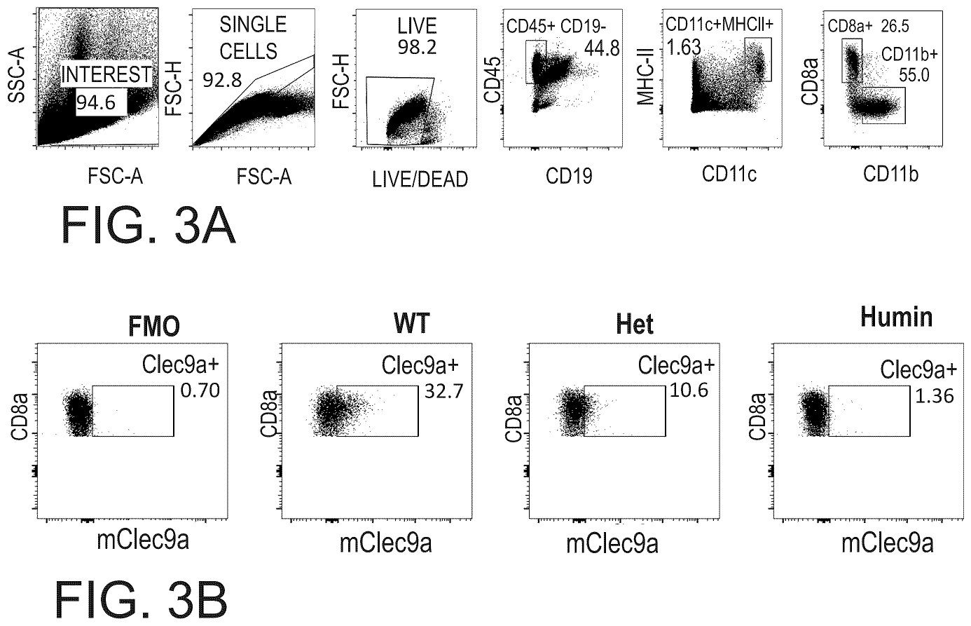

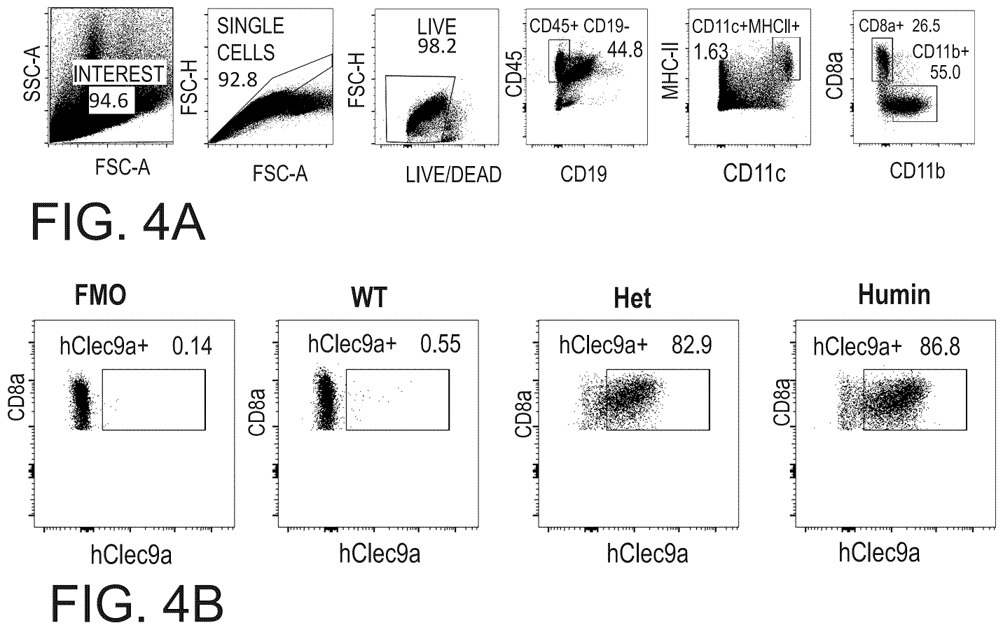

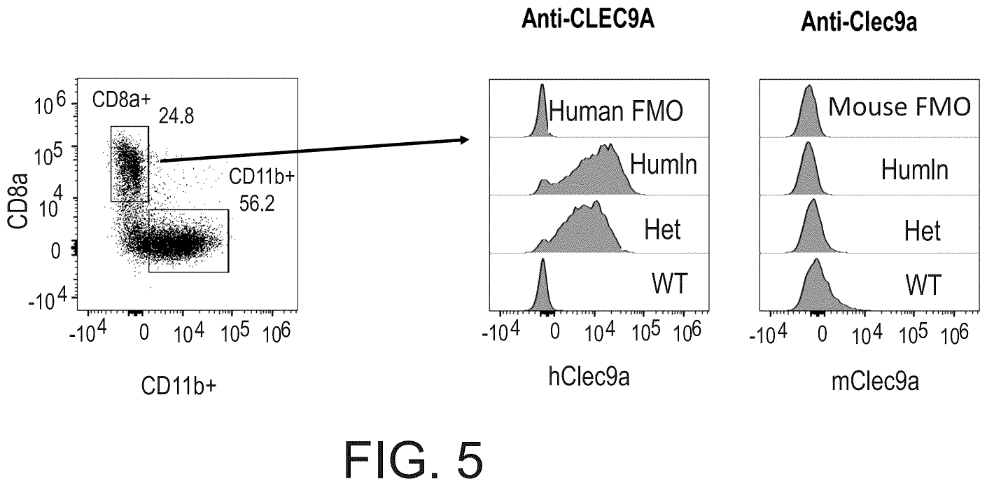

To ensure that humanized Clec9a was expressed, mice were injected with Flt3L to expand the dendritic cell population. Splenocytes were then isolated from wild-type (WT), heterozygous (Het), or homozygous (HumIn) mice and analyzed using FACS. Dendritic cells were gated based on MHC-II⁺ CD11c⁺ after gating on live and CD45⁺ cells and proceeded to gate on cDC1 (Xcr1⁺) cells (FIG. 3A). Next, cells expressing mouse Clec9a were sorted (FIG. 3B) and it was determined that mouse Clec9a was only expressed in wild-type and heterozygous mice, the latter carrying one copy of the murine Clec9a and one copy of humanized Clec9a. As expected, the HumIn mice homozygous for humanized Clec9a did not express the murine protein. Conversely, humanized Clec9a was detected on Xcr1⁺ DCs derived from the HumIn mice as well as the Het mice (FIG. 4B and FIG. 5). As expected, the wild-type mice did not express the humanized Clec9a protein.

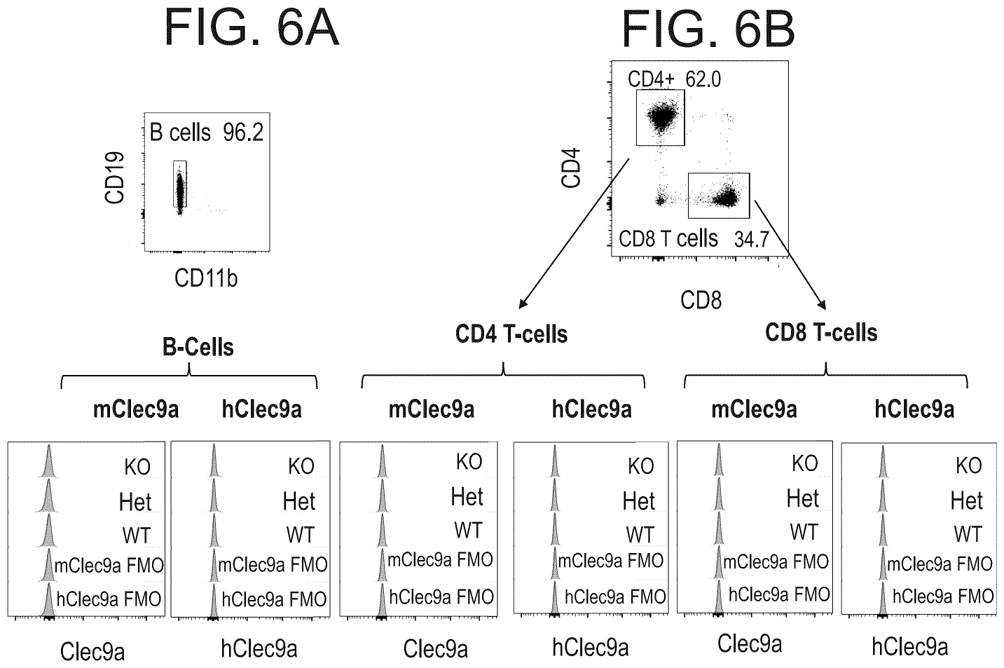

Next it was determined whether the genetic modifications (humanization of the endogenous Clec9a gene in mice) impacted expression beyond dendritic cells. To this end, B-cells (CD19⁺) (FIG. 6A) or CD4⁺ and CD8⁺ T-cells (FIG. 6B) were gated, and expression of either mouse or humanized Clec9a was probed. Expression of humanized Clec9a was not detected on the aforementioned cell populations indicating that expression was restricted to dendritic cells.

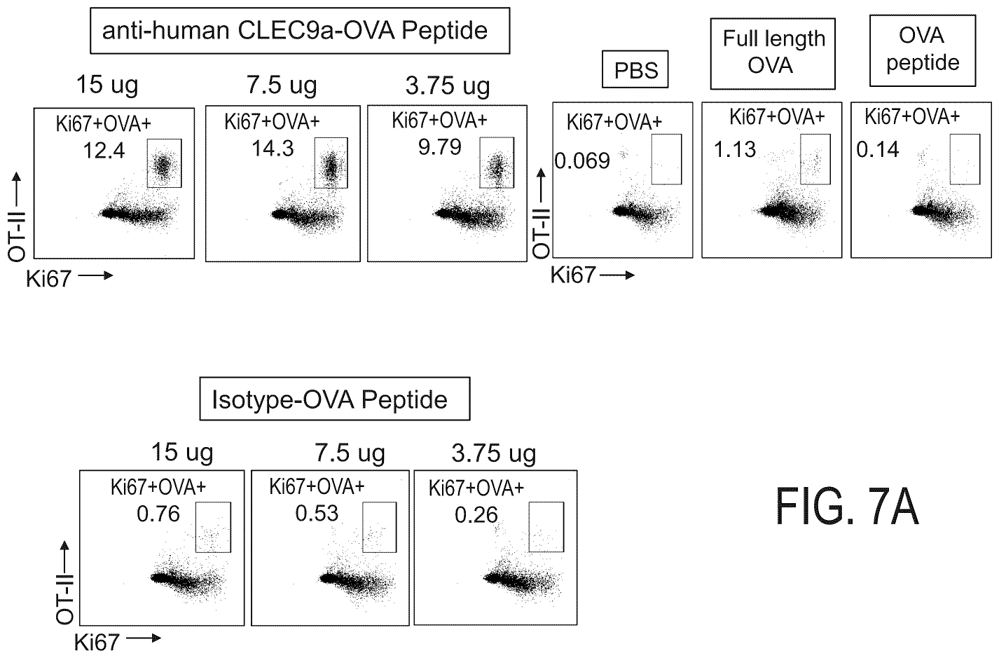

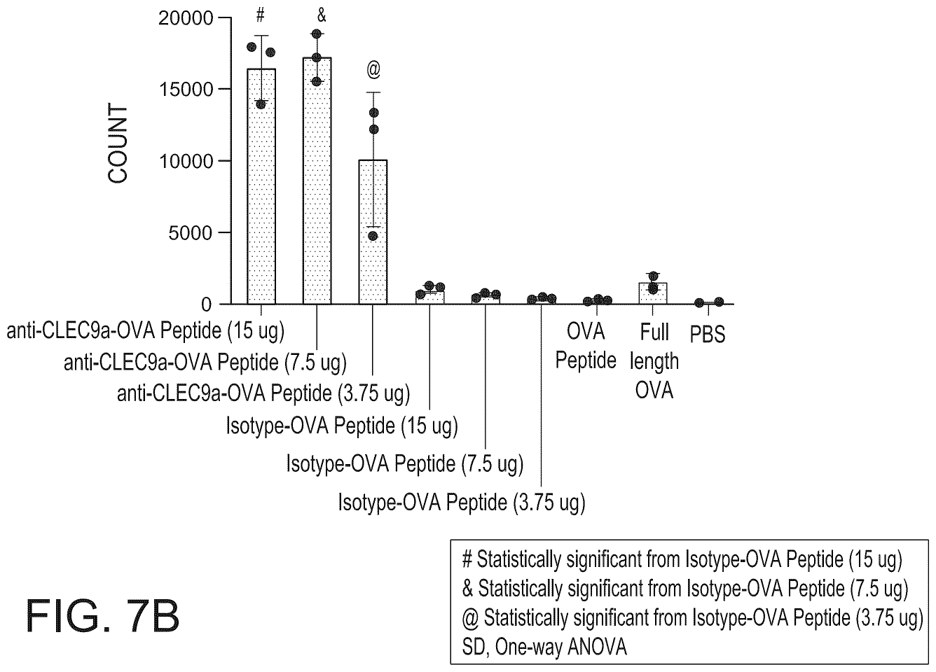

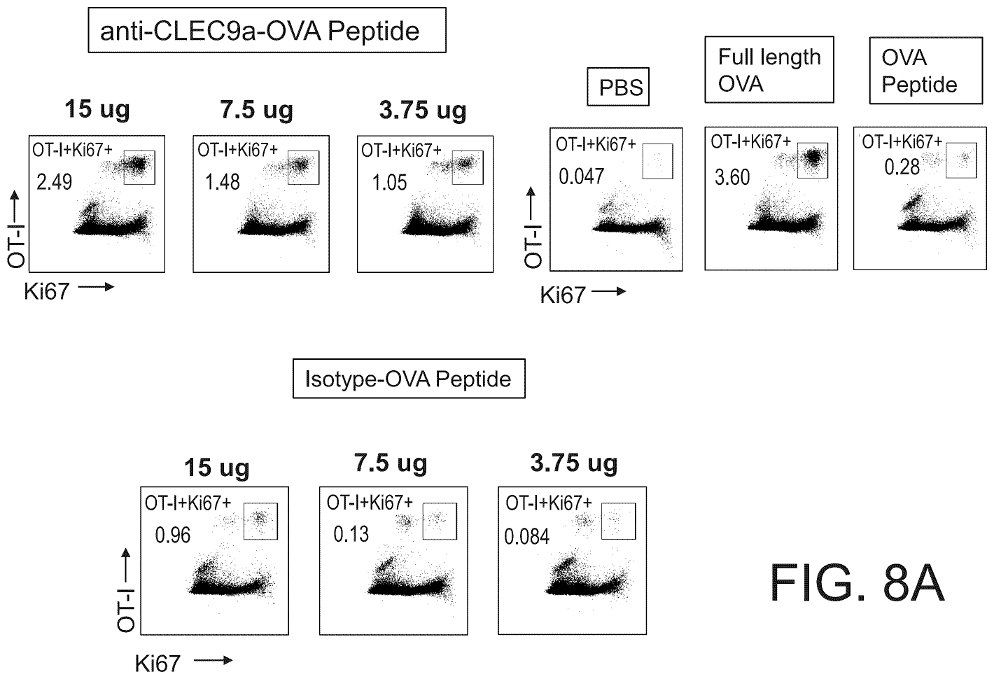

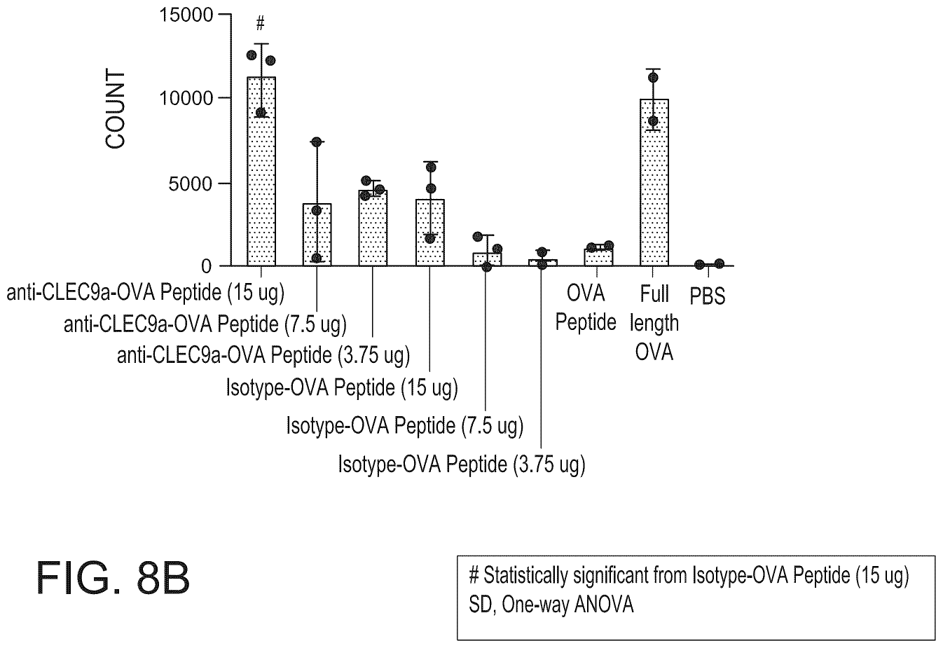

An in vivo proliferation assay was performed to determine whether humanized Clec9a functions in the genetically modified mice as intended. FIGS. 7A-7B and FIGS. 8A-8B demonstrate that targeting humanized Clec9a expressed on dendritic cells by using a fusion of an anti-human CLEC9a ectodomain antibody and a peptide containing OT-I and OT-II antigens elicited strong T-cell responses in vivo, indicating that the humanized Clec9a expressed on dendritic cells in the genetically modified mice is fully functional.

### Materials and Methods

Flt3L injections—Mice were injected with 10 ug of human Flt3L (purified in-house) daily for 5 days intraperitoneal in 100 uL of PBS.

Flow Cytometry—Spleens were processed in cold serum free RPMI 1640 and homogenized using the gentle MACS Dissociator instrument (Miltenyi, Cat #130-093-235). Spleen cell suspensions were mechanically disrupted through a 70 μm nylon cell strainer (Miltenyi, Cat. #130-110-916) with the back of a syringe plunger. Red blood cells (RBCs) were removed from the splenic single cell suspensions using ACK lysis buffer (Gibco, Cat. #A10492-01). Cells were stained with Live/Dead Blue-Fixable Blue Dye (eBioscience; Cat. #L23105) and mouse Fc Block (Biolegend, Cat. #101320) for 10 minutes at room temperature. The cells were washed twice in cell stain buffer (Biolegend, Cat. #420201). The following monoclonal antibodies were used to stain the cells in Brilliant Stain Buffer (BD Biosciences cat #566349) for thirty minutes on ice: CD45 BV510 (Clone 30-F11, Biolegend, Cat. #10138), CD11b BUV395 (Clone Ml/70, BD Bioscience, Cat. #563553), MHC-II Alexa Fluor 700 (Clone M5/114.152, Biolegend, Cat. #107622), CD11c Pe-Cy7 (Clone N418, Biolegend, Cat. #117318), F4/80 BV605 (Clone BM8, Cat. #123133), CD8a BUV805 (Clone 53-6.7, BD Bioscience, Cat. #612898), Xcr1 BV421 (Clone ZET, Biolegend, Cat. #148216), human Clec9a APC (Clone 8F9, Biolegend, Cat. #353806, directed to the ectodomain portion of the human CLEC9a protein), CD86 PerCP-Cy-5.5 (Clone GL-1, Biolegend, Cat. #105028), PDL1 BV711 (Clone 10.F.9G2, Cat. #124319), mouse Clec9a PE (Clone 42D2, eBioscience, Cat #L2129905, directed to the ectodomain of mouse Clec9a), Gr-1 FITC (Clone RB6-8C5, Biolegend, Cat. #108406, directed against Gr-1 which is a protein expressed on monocytes, granulocytes, and neutrophils), CD19 APC-Cy7 (Clone 6D5, Biolegend, Cat. #115530), CD4 BV786 (Clone RM4-5, BD Bioscience, Cat. #563727), and NK1.1 BV650 (Clone PKI36, BD Bioscience, Cat. #564143).

RNA Extraction and RT-PCR—Total RNA was purified using MagMAX™-96 for Microarrays Total RNA Isolation Kit Catalog #AM1839 (Ambion by Life Technologies) according to manufacturer's specifications. Genomic DNA was removed using MagMAX™Turbo™DNase Buffer and TURBO DNase from the MagMAX kit listed above (Ambion by Life Technologies). mRNA (Up to 2.5 ug) was reverse-transcribed into cDNA using SuperScript® VILO™ Master Mix Catalog #11755500 (Invitrogen by Life Technologies). cDNA was diluted to 0.5-5 ng/uL. 2.5-25 ng cDNA input was amplified with the SensiFAST Hi-ROX MasterMix (1×100 mL) Catalog #CSA-01113 (BIOLINE) using the ABI 7900HT Sequence Detection System (Applied Biosystems).

In vivo Proliferation Assay—CD4+ T cells or CD8+ T cells were purified from B6.Cg-Tg(TcraTcrb)425Cbn/mouse splenocytes using EasySep Mouse CD4+ or CD8+ T cell Isolation Kit (StemCell Tech; Cat. 19852 for CD4+ T cell isolation and Cat. 19853 for CD8+ T cell isolation). These purified T cells were then labeled with CFSE cell trace dye (Invitrogen; Cat. #C34554A). 2 E+06 cells were injected retro-orbitally in each HumIn mouse expressing humanized Clec9a. Twenty-four hours later, the mice received the following treatment groups subcutaneously: 15 ug anti-hCLEC9a-Peptide (n=3), 7.5 ug anti-hCLEC9a-Peptide (n=3), 3.75 ug anti-hCLEC9a-Peptide (n=3). The anti-hCLEC9a-Peptide is an antibody-peptide fusion, wherein the antibody is directed to the ectodomain of human CLEC9a and the peptide comprises the OT-I (amino acids 257-264 of OVA) and OT-II (amino acids 323-339 of OVA) antigens. The isotype-peptide fusion (i.e., fusion between an isotype antibody and the peptide comprising the OT-I and OT-II antigens) was dosed in a similar fashion. A separate group of mice received 5 mg/kg Ovalbumin EndoFit ((n=3) InvivoGen; cat. #vac-pova), and 0.03366 mg/kg of peptide alone (n=3). 72 hours later, the mice were euthanized and spleens were harvested and purified for flow.

1. A genetically modified rodent animal comprising in its genome:
a humanized Clec9a gene comprising:
a rodent Clec9a nucleic acid sequence, and
a human CLEC9A nucleic acid sequence,
wherein the humanized Clec9a gene encodes a humanized Clec9a polypeptide comprising an ectodomain at least 90% identical in sequence to the ectodomain of a human CLEC9A protein, and
wherein the rodent animal is a mouse or a rat.

2. (canceled)

3. The genetically modified rodent animal of claim 1, wherein the human CLEC9A protein comprises the amino acid sequence as set forth in SEQ ID NO: 4.

4. The genetically modified rodent animal of claim 1, wherein the humanized Clec9a protein comprises a cytoplasmic-transmembrane sequence at least 90% identical in sequence to the cytoplasmic-transmembrane sequence of a rodent Clec9a protein.

5. (canceled)

6. The genetically modified rodent animal of claim 4, wherein the rodent Clec9a protein is an endogenous rodent Clec9a protein.

7. The genetically modified rodent animal of claim 1, wherein the human CLEC9A nucleic acid sequence comprises exon 3 through exon 6 of a human CLEC9A gene.

8. The genetically modified rodent animal of claim 1, wherein the rodent Clec9a nucleic acid sequence comprises exons 1 and 2 of a rodent Clec9a gene and/or the 3′ untranslated region of exon 6 of the rodent Clec9a gene.

9. (canceled)

10. The genetically modified rodent animal of claim 8, wherein the rodent Clec9a gene is an endogenous rodent Clec9a gene.

11. (canceled)

12. The genetically modified rodent animal of claim 1, wherein the humanized Clec9a gene is operably linked to a rodent Clec9a promoter.

13. The genetically modified rodent animal of claim 12, wherein the rodent Clec9a promoter is the endogenous rodent Clec9a promoter.

14. The genetically modified rodent animal of claim 1, wherein the humanized Clec9a gene is located at an endogenous rodent Clec9a locus.

15. The genetically modified rodent animal of claim 14, wherein the humanized Clec9a gene is formed as a result of replacement of a rodent Clec9a genomic DNA at an endogenous rodent Clec9a locus with the human CLEC9A nucleic acid.

16. The genetically modified rodent animal of claim 15, wherein the humanized Clec9a gene is formed as a result of replacement of a rodent genomic DNA comprising exon 3 through the Stop codon in exon 6 of an endogenous rodent Clec9a gene with the human CLEC9A nucleic acid, and wherein the human CLEC9A nucleic acid comprises exon 3 through exon 6 of a human CLEC9A gene.

17. The genetically modified rodent animal of claim 1, wherein the rodent is heterozygous for the humanized Clec9a gene.

18. The genetically modified rodent animal of claim 1, wherein the rodent is homozygous for the humanized Clec9a gene.

19. The genetically modified rodent animal of claim 1, wherein the rodent expresses the humanized Clec9a polypeptide on dendritic cells.

20. (canceled)

21. The genetically modified rodent animal of claim 1, wherein the rodent animal is a mouse and the humanized Clec9a polypeptide comprises the amino acid sequence as set forth in SEQ ID NO: 7.

22. An isolated rodent tissue or cell, whose genome comprises a humanized Clec9a gene comprising a rodent Clec9a nucleic acid sequence and a human CLEC9A nucleic acid sequence, wherein the humanized Clec9a gene encodes a humanized Clec9a polypeptide comprising an ectodomain at least 90% identical in sequence to the ectodomain of a human CLEC9A protein, and wherein the rodent tissue or cell is a mouse tissue or cell, or a rat tissue or cell.

23. The isolated rodent tissue or cell of claim 22, wherein the rodent cell is a rodent embryonic stem cell.

24. The isolated rodent tissue or cell of claim 22, wherein the rodent cell is a rodent germ cell.

25.-26. (canceled)

27. The isolated rodent tissue or cell of claim 22, wherein the humanized Clec9a protein comprises a cytoplasmic-transmembrane sequence at least 90% identical to the cytoplasmic-transmembrane sequence of a rodent Clec9a protein.

28.-29. (canceled)

30. The isolated rodent tissue or cell of claim 22, wherein the rodent tissue or cell is a mouse tissue or cell, and the humanized Clec9a protein comprises the amino acid sequence of SEQ ID NO: 7.

31. A rodent embryo, comprising the rodent embryonic stem cell of claim 23.

32. A method of making a genetically modified rodent animal, comprising:
modifying a rodent genome to comprise a humanized Clec9a gene, wherein the humanized Clec9a gene comprises a rodent Clec9a nucleic acid sequence and a human CLEC9A nucleic acid sequence, and encodes a humanized Clec9a polypeptide comprising an ectodomain at least 90% identical in sequence with the ectodomain of a human CLEC9A protein, and wherein the rodent animal is a mouse or a rat; and
making the rodent animal comprising the modified rodent genome.

33. The method of claim 32, wherein said modifying comprises
introducing a nucleic acid molecule comprising the human CLEC9A nucleic acid sequence into the genome of a rodent embryonic stem (ES) cell,
obtaining a rodent ES cell in which the human CLEC9A nucleic acid sequence has been integrated into an endogenous Clec9a locus to replace a rodent Clec9a genomic DNA thereby forming the humanized Clec9a gene, and
generating a rodent animal from the obtained rodent ES cell.

34.-45. (canceled)

46. A targeting nucleic acid construct, comprising
a human CLEC9A nucleic acid sequence to be integrated into a rodent Clec9a gene at an endogenous rodent Clec9a locus, flanked by a 5′ nucleotide sequence and a 3′ nucleotide sequence that are homologous to nucleotide sequences at the rodent Clec9a locus,
wherein integration of the human CLEC9A nucleic acid sequence into the rodent Clec9a gene results in a replacement of a rodent Clec9a genomic DNA with the human CLEC9A nucleic acid sequence thereby forming a humanized Clec9a gene, and
wherein the humanized Clec9a gene encodes a humanized Clec9a polypeptide comprising an ectodomain substantially identical with the ectodomain of a human CLEC9A protein.

47.-50. (canceled)

51. A method of assessing pharmacokinetic properties of a candidate drug, comprising:
administering the candidate drug to a rodent animal according to claim 1;
performing one or more assays to determine the pharmacokinetic properties of the candidate drug in the rodent animal.

52. A method for screening candidate drugs targeting human CLEC9A, comprising:
administering the candidate drug to a rodent animal according to claim 1; and
performing one or more assays to determine whether the candidate drug has an effect on the rodent animal.

53.-56. (canceled)
